(19) United States
(12) Patent Application Publication
(10) Pub. No.: US 2012/0141381 A1
(43) Pub. Date: Jun. 7, 2012

(54) Methods For Loading Contrast Agents Into A Liposome

(75) Inventors: Mark W. Dewhirst, Durham, NC (US); Pavel Yarmolenko, Chapel Hill, NC (US); Bradford J. Wood, Potomac, MD (US); Matthrew R. Dreher, Rockville, MD (US); Ayele H. Negussie, Silver Spring, MD (US)

(73) Assignee: Duke University Office of Technology & Venture, Durham, NC (US)

(21) Appl. No.: 13/202,920

(22) PCT Filed: Feb. 23, 2010
(86) PCT No.: PCT/US10/25049

§ 371 Date: Feb. 29, 2012

Related U.S. Application Data
(60) Provisional application No. 61/154,663, filed on Feb. 23, 2009.

(51) Int. Cl. A61K 49/18

(52) U.S. Cl. 424/9.321; 264/4.3

(57) ABSTRACT

The invention comprises compositions and methods for loading both magnetic resonance contrast agents and therapeutic agents into liposomes, such as low temperature sensitive liposomes (LTSLs). In certain embodiments, a passive technique is used to load the liposomes. In other embodiments, an active technique is used to load the liposomes. In further embodiments, a magnetic resonance contrast agent and Doxorubicin are loaded into the liposomes. The liposome compositions have higher contrast-agent loadings and are more stable, than those known in the art.

### RELATED APPLICATION

This application claims the benefit of priority to U.S. Provisional Patent Application Ser. No. 61/154,663, filed Feb. 23, 2009; the contents of which are hereby incorporated by reference.

### BACKGROUND

General techniques for liposomal delivery of therapeutic agents is disclosed in the following publications, which are incorporated by reference: Tilcok C P; Ahkong Q F; Parr M. Investigative Radiology, 1991, 26, 242-7; Ponce, A M.; Viglianti, B L.; Yu, D.; Yarmolenko, P S.; Michelich, C R.; Woo, J.; Bally, M B.; Dewhirst, M W. Journal of the National Cancer Institute, 2007, 99, 53-6; Uyen M L.; Cui, Z. International Journal of Pharmaceutics, 2006, 312, 10 105-12; Laurent, S.; Elst, L V.; Thirifays, C.; Muller, R N. Langmuir, 2008, 24, 4347-51; Le, U. M.; Shaker, D S.; Sloat, B, R.; Cui, Z. Drug Development and Industrial Pharmacy, 2008, 34, 413-8; Kamaly, N.; Kalber, T.; Ahmad, A.; Oliver, M H.; So, P-W.; Herlihy, A H.; Bell, J. D.; Jorgensen, M R.; Miller A D. Bioconjugate Chemistry, 2008, 19, 118-29; Glogard, C.; Stensrud, G.; Aime, S. Magnetic Resonance in Chemistry, 2003, 41, 585-8; and Saito, R.; Krauze, M T.; Bringas, J R.; Noble, C; McKnight, R. T.; Jackson, P.; Wenland, M F. ; Mamot, C; Crummond, D C.; Kirpotin, D B., Hong, K.; Berger, M S.; Park, J W.; Bankiewicz, K S. Experimental Neurology, 2005, 196, 381-9.

MRI image-able liposomes have been in development for nearly two decades. Several liposomal nanocarriers with image-able components have been investigated to discern pathologic sites, to allow blood pool imaging, to determine tumor vascular permeability, to visualize important vascular features, to map radicals and to report on drug delivery (Caravan et al. (1999) Chemical Reviews 99(9):2293-352; Karathanasis et al. (2008) Nanotechnology 19(31); Ghaghada et al. (2007) American Journal of Neuroradiology 28(1):48-53; and Glogard et al. (2003) Magnetic Resonance in Chemistry 41(8):585-8). While manganese has been used to report on drug delivery with liposomes (see, for example, PCT Application No. PCT/US2007/026493, hereby incorporated by reference in its entirety), gadolinium-based contrast agents are likely better candidates due to their greater acceptance in the clinic, and therefore much of the recent work on magnetic resonance image-able liposomes involves Gd-based contrast agents.

In addition to the choice of contrast agent, the type of association of the contrast agent with the liposome impacts utility of image-able liposomes. Gd-based contrast agents have been incorporated into liposome core as well as conjugated to their membrane or both (Tilcock et al. (1989) Radiology 171(1):77-80; Ghaghada et al. (2008) Academic Radiology. 15(10):1259-63; and Mulder et al. (2004) Bioconjugate Chemistry 15(4):799-806). Among these different loading methods, liposomes with contrast agent on their surface offer exceptionally high relaxivity, likely due to high rotational correlation times of surface-bound contrast agents. Therefore, liposomes with contrast agent conjugated to the membrane may be promising for tumor microenvironment studies. However, such formulations have not been shown useful for reporting on drug release, where liposomes with contrast agent in the lumen have shown promising correlation with drug delivery and efficacy.

Low temperature sensitive liposomes (LTSLs) are heat activated liposomal agents that selectively and safely release their payload in a region of the body that is heated above body temperature (about 41° C.). Therefore, LTSLs are capable of selective delivery of therapeutic agents to specific regions of the body. Magnetic resonance-guided high intensity focus ultrasound (MRgHIFU) is capable of selectively heating specific regions of the body with magnetic resonance-guidance and temperature feedback. The ability to monitor drug release in the magnetic resonance environment is an enabling technology that is positioned to change the way drugs are delivered to tumors using MRgHIFU. A dose of chemotherapeutic agent may be selectively painted in a desired region of the body, such as a tumor, with real-time feedback to improve the therapy. This technology may be applied to other conditions such as atherosclerosis and thrombosis.

Contrast agent loaded liposomes have previously been used to indicate drug release in the magnetic resonance environment and serve as a surrogate for drug delivery. However, reported methods for loading contrast agents inside liposomes can be prohibitively expensive, toxic, and/or not performed in conjunction with therapeutic agents.

### SUMMARY

One aspect of the invention relates to methods for loading both magnetic resonance contrast agents and therapeutic agents into liposomes, such as low temperature sensitive liposomes (LTSLs). In certain embodiments, a passive technique is used to load the liposomes. In other embodiments, an active technique is used to load the liposomes. In certain embodiments, a magnetic resonance contrast agent and Doxorubicin are loaded into the liposomes. In certain embodiments, these liposome compositions have higher contrast-agent loadings, and are more stable, than those previously reported.

One aspect of the invention relates to a method comprising the steps of: reconstituting liposome-forming lipids with a solution comprising a contrast agent and a hydrating buffer, wherein the hydrating buffer has an osmolarity of between about 300 mOsm and about 700 mOsm; incubating the pre-liposome solution at a temperature for a time; and extruding the incubated solution through a filter, thereby forming a liposome.

In certain embodiments, the present invention relates to any of the aforementioned methods, wherein the liposome-forming lipids comprise phospholipids.

In certain embodiments, the present invention relates to any of the aforementioned methods, wherein the liposome-forming lipids comprise phosphatidylcholines. In certain embodiments, the present invention relates to any of the aforementioned methods, wherein the liposome-forming lipids comprise phosphatidylcholines selected from the group consisting of dipalmitoylphosphatidylcholine (DPPC), distearoylphosphatidylcholine (DSPC), monostearoylphosphatidylcholine (MSPC), diarachidoylphosphatidylcholine (DAPC), and mixtures thereof. In certain embodiments, the present invention relates to any of the aforementioned methods, wherein the liposome-forming lipids comprise dipalmitoylphosphatidylcholine, monostearoylphosphatidylcholine, and 1,2-distearoyl-sn-glycero-3-phosphoethanolamine-N-[Amino(Polyethylene Glycol)2000]. In certain embodiments, the present invention relates to any of the aforementioned methods, wherein the liposome-forming lipids comprise about 85.3 mol % dipalmitoylphosphatidylcholine, about 9.7 mol % monostearoylphosphatidylcholine, and about 5.0 mol % 1,2-distearoyl-sn-glycero-3-phosphoethanolamine-N-[amino(polyethylene glycol)2000].

In certain embodiments, the present invention relates to any of the aforementioned methods, wherein the liposome is a non-sensitive liposome. In certain embodiments, the present invention relates to any of the aforementioned methods, wherein the liposome is an envirosensitive liposome. In certain embodiments, the present invention relates to any of the aforementioned methods, wherein the envirosensitve liposome is selected from the group consisting of thermosensitive liposome, a pH-sensitive liposome, a chemosensitive liposome, radiation-sensitive liposome and combinations thereof.

In certain embodiments, the present invention relates to any of the aforementioned methods, wherein instead of forming a liposome, a polymersome is used. For example, in certain embodiments, the “liposome-forming lipids” comprise amphiphilic polymers, which form a polymersome.

In certain embodiments, the present invention relates to any of the aforementioned methods, wherein the contrast agent comprises an element selected from the group consisting of Gd, Cu, Cr, Fe, Co, Er, Ni, Eu, Dy, Zn, Mg, Mo, Li, Ta, and Mn. In certain embodiments, the present invention relates to any of the aforementioned methods, wherein the contrast agent comprises Gd. In certain embodiments, the present invention relates to any of the aforementioned methods, wherein the contrast agent is gadopentetate dimeglumine. In certain embodiments, the present invention relates to any of the aforementioned methods, wherein the contrast agent is gadoteridol.

In certain embodiments, the present invention relates to any of the aforementioned methods, wherein the concentration of the contrast agent in solution is between about 100 mM and about 500 mM, about 100 mM and about 200 mM, about 200 mM and about 300 mM, about 300 mM and about 400 mM, or about 400 mM and about 500 mM. In certain embodiments, the present invention relates to any of the aforementioned methods, wherein the concentration of the contrast agent in the liposome is between about 200 mM and about 500 mM, about 250 mM and about 450 mM, or about 300 mM and about 400 mM.

In certain embodiments, the present invention relates to any of the aforementioned methods, wherein the concentration of the contrast agent in the liposome is about 300 mM. In certain embodiments, the present invention relates to any of the aforementioned methods, wherein the concentration of the contrast agent in the liposome is about 300 mM; and the contrast agent is gadoteridol. In certain embodiments, the present invention relates to any of the aforementioned methods, wherein the concentration of the contrast agent in the liposome is about 300 mM; and the contrast agent is gadoteridol; and the diameter of the liposome is about 100 nm. As shown in FIG. 9A, keeping ProHance to a concentration of less than 300 mM maintains a stable particle.

In certain embodiments, the present invention relates to any of the aforementioned methods, wherein the osmolarity of the hydrating buffer is between about 400 mOsm and about 700 mOsm, about 500 mOsm and about 700 mOsm, about 400 mOsm and about 600 mOsm, about 400 mOsm and about 500 mOsm, about 500 mOsm and about 500 mOsm, or about 600 mOsm and about 700 mOsm.

In certain embodiments, the present invention relates to any of the aforementioned methods, wherein the hydrating buffer comprises citrate. In certain embodiments, the present invention relates to any of the aforementioned methods, wherein the concentration of citrate in the hydrating buffer is between about 75 mM and about 300 mM, or about 150 mM and about 250 mM. In certain embodiments, the present invention relates to any of the aforementioned methods, wherein the concentration of citrate in the hydrating buffer is about 90 mM, about 150 mM, or about 250 mM.

In certain embodiments, the present invention relates to any of the aforementioned methods, wherein the temperature is between about 40° C. and about 70° C. In certain embodiments, the present invention relates to any of the aforementioned methods, wherein the temperature is between about 40° C. and about 50° C. In certain embodiments, the present invention relates to any of the aforementioned methods, wherein the temperature is between about 50° C. and about 60° C. In certain embodiments, the present invention relates to any of the aforementioned methods, wherein the temperature is between about 60° C. and about 70° C. In certain embodiments, the present invention relates to any of the aforementioned methods, wherein the temperature is 55° C. In certain embodiments, the present invention relates to any of the aforementioned methods, wherein the temperature is 60° C.

In certain embodiments, the present invention relates to any of the aforementioned methods, wherein the time is between about 5 minutes and about 60 minutes. In certain embodiments, the present invention relates to any of the aforementioned methods, wherein the time is about 10 minutes. In certain embodiments, the present invention relates to any of the aforementioned methods, wherein the time is about 20 minutes. In certain embodiments, the present invention relates to any of the aforementioned methods, wherein the time is about 30 minutes. In certain embodiments, the present invention relates to any of the aforementioned methods, wherein the time is about 60 minutes.

In certain embodiments, the present invention relates to any of the aforementioned methods, wherein the filter is a polycarbonate membrane filter. In certain embodiments, the present invention relates to any of the aforementioned methods, wherein the filter has a pore size of about 100 nm, 150 nm, 200 nm, 250 nm or 300 nm.

In certain embodiments, the present invention relates to any of the aforementioned methods, further comprising the steps of: neutralizing the outside pH of the liposome; and contacting the neutralized liposome with a compound under conditions wherein the compound is encapulsated by the liposome.

In certain embodiments, the present invention relates to any of the aforementioned methods, wherein the compound is a chemotherapeutic agent. In certain embodiments, the present invention relates to any of the aforementioned methods, wherein the chemotherapeutic agent is Doxorubicin.

In certain embodiments, the present invention relates to any of the aforementioned methods, wherein the step of neutralzing the outside pH of the liposome comprises contacting the liposome with an external buffer via a buffer exchange. In certain embodiments, the present invention relates to any of the aforementioned methods, wherein the external buffer has an osmolality of between about 200 mOsm and about 700 mOsm, about 200 mOsm and about 300 mOsm, about 300 mOsm and about 400 mOsm, about 400 mOsm and about 500 mOsm, about 500 mOsm and about 600 mOsm, about 600 mOsm and about 700 mOsm, or about 200 mOsm and about 400 mOsm. In certain embodiments, the present invention relates to any of the aforementioned methods, wherein the external buffer has a pH of between about 7 and about 8. In certain embodiments, the present invention relates to any of the aforementioned methods, wherein the external buffer comprises 0.5M of Na₂CO₃, and has a pH of about 7.5.

Another aspect of the invention relates to a method comprising the steps of: reconstituting liposome-forming lipids with a solution comprising a chelating agent and a hydrating buffer; incubating the pre-liposome solution at a temperature for a time; extruding the incubated solution through a filter, thereby forming a liposome comprising the chelating agent; contacting the liposome comprising the chelating agent with an external buffer, thereby neutralizing the outside pH of the liposome; contacting the neutralized liposome with a compound under conditions wherein the compound is encapulsated by the liposome, thereby forming a liposome comprising the compound and the chelating agent; and contacting the liposome comprising the compound and the chelating agent with an ionophore and a metal ion, under conditions where the ionophore assists in the encapuslation of the metal ion by the liposome comprising a compound and a chelating agent, thereby forming a liposome.

In certain embodiments, the present invention relates to any of the aforementioned methods, wherein the liposome-forming lipids comprise phospholipids. In certain embodiments, the present invention relates to any of the aforementioned methods, wherein the liposome-forming lipids comprise phosphatidylcholines. In certain embodiments, the present invention relates to any of the aforementioned methods, wherein the liposome-forming lipids comprise phosphatidylcholines selected from the group consisting of dipalmitoylphosphatidylcholine (DPPC), distearoylphosphatidylcho line (DSPC), monostearoylphosphatidylcho line (MSPC), diarachidoylphosphatidylcho line (DAPC), and mixtures thereof. In certain embodiments, the present invention relates to any of the aforementioned methods, wherein the liposome-forming lipids comprise dipalmitoylphosphatidylcholine, monostearoylphosphatidylcholine, and 1,2-distearoyl-sn-glycero-3-phosphoethanolamine-N-[Amino(Polyethylene Glycol)2000]. In certain embodiments, the present invention relates to any of the aforementioned methods, wherein the liposome-forming lipids comprise about 85.3 mol % dipalmitoylphosphatidylcholine, about 9.7 mol % monostearoylphosphatidylcholine, and about 5.0 mol % 1,2-distearoyl-sn-glycero-3-phosphoethano lamine-N-[amino(polyethylene glycol)2000].

In certain embodiments, the present invention relates to any of the aforementioned methods, wherein the liposome is a non-sensitive liposome. In certain embodiments, the present invention relates to any of the aforementioned methods, wherein the liposome is an envirosensitive liposome. In certain embodiments, the present invention relates to any of the aforementioned methods, wherein the envirosensitve liposome is selected from the group consisting of a thermosensitive liposome, a pH-sensitive liposome, a chemosensitive liposome, a radiation-sensitive liposome and combinations thereof.

In certain embodiments, the present invention relates to any of the aforementioned methods, wherein instead of forming a liposome, a polymersome is used. For example, in certain embodiments, the “liposome-forming lipids” comprise amphiphilic polymers, which form a polymersome.

In certain embodiments, the present invention relates to any of the aforementioned methods, wherein the chelating agent is diethylene triamine pentaacetic acid (DTPA). In certain embodiments, the present invention relates to any of the aforementioned methods, wherein the concentration of the chelating agent in solution is between about 100 mM and about 500 mM, about 100 mM and about 200 mM, about 200 mM and about 300 mM, about 300 mM and about 400 mM, or about 400 mM and about 500 mM. In certain embodiments, the present invention relates to any of the aforementioned methods, wherein the concentration of the chelating agent in the liposome is between about 100 mM and about 500 mM, about 100 mM and about 200 mM, about 200 mM and about 300 mM, about 300 mM and about 400 mM, or about 400 mM and about 500 mM.

In certain embodiments, the present invention relates to any of the aforementioned methods, wherein the osmolarity of the hydrating buffer is between about 400 mOsm and about 700 mOsm, about 500 mOsm and about 700 mOsm, about 400 mOsm and about 600 mOsm, about 400 mOsm and about 500 mOsm, about 500 mOsm and about 500 mOsm, or about 600 mOsm and about 700 mOsm.

In certain embodiments, the present invention relates to any of the aforementioned methods, wherein the hydrating buffer comprises citrate. In certain embodiments, the present invention relates to any of the aforementioned methods, wherein the concentration of citrate in the hydrating buffer is between about 75 mM and about 300 mM, or about 150 mM and about 250 mM. In certain embodiments, the present invention relates to any of the aforementioned methods, wherein the concentration of citrate in the hydrating buffer is about 90 mM, about 150 mM, or about 250 mM.

In certain embodiments, the present invention relates to any of the aforementioned methods, wherein the temperature is between about 40° C. and about 70° C. In certain embodiments, the present invention relates to any of the aforementioned methods, wherein the temperature is between about 40° C. and about 50° C. In certain embodiments, the present invention relates to any of the aforementioned methods, wherein the temperature is between about 50° C. and about 60° C. In certain embodiments, the present invention relates to any of the aforementioned methods, wherein the temperature is between about 60° C. and about 70° C. In certain embodiments, the present invention relates to any of the aforementioned methods, wherein the temperature is 55° C. In certain embodiments, the present invention relates to any of the aforementioned methods, wherein the temperature is 60° C.

In certain embodiments, the present invention relates to any of the aforementioned methods, wherein the time is between about 5 minutes and about 60 minutes. In certain embodiments, the present invention relates to any of the aforementioned methods, wherein the time is about 10 minutes. In certain embodiments, the present invention relates to any of the aforementioned methods, wherein the time is about 20 minutes. In certain embodiments, the present invention relates to any of the aforementioned methods, wherein the time is about 30 minutes. In certain embodiments, the present invention relates to any of the aforementioned methods, wherein the time is about 60 minutes.

In certain embodiments, the present invention relates to any of the aforementioned methods, wherein the filter is a polycarbonate membrane filter. In certain embodiments, the present invention relates to any of the aforementioned methods, wherein the filter has a pore size of about 100 nm, about 150 nm, about 200 nm, about 250 nm, or about 300 nm.

In certain embodiments, the present invention relates to any of the aforementioned methods, wherein the step of contacting the liposome with an external buffer is a buffer exchange. In certain embodiments, the present invention relates to any of the aforementioned methods, wherein the external buffer has an osmolality of between about 200 mOsm and about 700 mOsm, about 200 mOsm and about 300 mOsm, about 300 mOsm and about 400 mOsm, about 400 mOsm and about 500 mOsm, about 500 mOsm and about 600 mOsm, about 600 mOsm and about 700 mOsm, or about 200 mOsm and about 400 mOsm In certain embodiments, the present invention relates to any of the aforementioned methods, wherein the external buffer has a pH of between about 7 and about 8. In certain embodiments, the present invention relates to any of the aforementioned methods, wherein the external buffer comprises 250 mM sucrose and 20 mM HEPES, and has a pH of about 7.5.

In certain embodiments, the present invention relates to any of the aforementioned methods, wherein the compound is a chemotherapeutic agent. In certain embodiments, the present invention relates to any of the aforementioned methods, wherein the chemotherapeutic agent is Doxorubicin.

In certain embodiments, the present invention relates to any of the aforementioned methods, wherein the ionophore is an ionophoretic antibiotic. In certain embodiments, the present invention relates to any of the aforementioned methods, wherein the ionophore is A23187.

In certain embodiments, the present invention relates to any of the aforementioned methods, wherein the metal ion comprises an element selected from the group consisting of Gd, Cu, Cr, Fe, Co, Er, Ni, Eu, Dy, Zn, Mg, Mo, Li, Ta, and Mn. In certain embodiments, the present invention relates to any of the aforementioned methods, wherein the metal ion is Gd(III).

Another aspect of the invention relates to a liposome prepared by any of the aforementioned methods. Another aspect of the invention relates to a polymersome prepared by any of the aforementioned methods.

Another aspect of the invention relates to a method of predicting efficacy of a treatment in a subject, the method comprising: administering to the subject a composition comprising a liposome prepared by any of the aforementioned methods, provided the liposome comprises a compound; monitoring accumulation of the compound at a desired site in vivo by magnetic resonance imaging; and predicting efficacy of treatment based on accumulation of the compound at the desired site.

In certain embodiments, the present invention relates to any of the aforementioned methods, wherein the liposome comprises DSPC/Cholesterol (55:45, mol:mol), DPPC-PEG₂₀₀₀, DPPC-DSPE-PEG₂₀₀₀ (95:5, mol:mol), or DPPC-MSPC-DSPE-PEG₂₀₀₀ (90:10:4, mol:mol).

In certain embodiments, the present invention relates to any of the aforementioned methods, further comprising exposing the liposome at the desired site to a non-physiological environmental condition.

In certain embodiments, the present invention relates to any of the aforementioned methods, wherein the environmental condition is selected from the group consisting of hyperthermia, electromagnetic radiation, a chemical agent and non-physiological pH.

In certain embodiments, the present invention relates to any of the aforementioned methods, wherein the desired site is selected from the group consisting of a tumor, an embolism, an injury site, an ischemia, and a tissue edema.

In certain embodiments, the present invention relates to any of the aforementioned methods, wherein monitoring the accumulation of the compound of interest at the desired site by magnetic resonance imaging comprises making a pixel density determination.

In certain embodiments, the present invention relates to any of the aforementioned methods, wherein predicting efficacy comprises predicting efficacy of treatment based on a location of accumulation at the desired site, a rate of accumulation at the desired site, or both location and rate of accumulation at the desired site.

Another aspect of the invention relates to a method of enhancing efficacy of a treatment at a desired site in a subject, the method comprising: administering to the subject a composition comprising a liposome prepared by any of the aforementioned methods, provided the liposome comprises a compound; and targeting the composition to a desired location at a desired site in the subject, at a desired rate of accumulation at the desired site, or both a desired location and a desired rate of accumulation at the desired site, to thereby enhance efficacy of treatment provided by the compound.

In certain embodiments, the present invention relates to any of the aforementioned methods, wherein the liposome comprises DSPC/Cholesterol (55:45, mol:mol), DPPC-PEG₂₀₀₀, DPPC-DSPE-PEG₂₀₀₀ (95:5, mol:mol), or DPPC-MSPC-DSPE-PEG₂₀₀₀ (90:10:4, mol:mol).

In certain embodiments, the present invention relates to any of the aforementioned methods, wherein a non-physiological environmental condition is present at the desired site, and the composition is targeted to a desired location at the desired site in the subject, at a desired rate of accumulation at the desired site, or both a desired location and a desired rate of accumulation at the desired site by the presence of the non-physiological environmental condition.

In certain embodiments, the present invention relates to any of the aforementioned methods,wherein the non-physiological environmental condition is selected from the group consisting of hyperthermia, electromagnetic radiation, a chemical agent and non-physiological pH.

In certain embodiments, the present invention relates to any of the aforementioned methods, wherein the non-physiological environmental condition is hyperthermia; and the hyperthermia is provided by a natural process or by a method selected from the group consisting of contacting a heated material with the desired site, applying RF energy, applying microwave energy to the desired site, applying ultrasonic energy to the desired site and applying a laser beam to the desired site.

In certain embodiments, the present invention relates to any of the aforementioned methods, wherein the desired site is exposed to a non-physiological environmental condition before, after, or both before and after administering the composition.

In certain embodiments, the present invention relates to any of the aforementioned methods, comprising administering the composition in one or more partial doses before, after, or both before and after the desired site is exposed to a non-physiological environmental condition.

In certain embodiments, the present invention relates to any of the aforementioned methods, wherein the desired site is selected from the group consisting of a tumor, an embolism, an injury site, an ischemia, and at a tissue edema.

In certain embodiments, the present invention relates to any of the aforementioned methods, further comprising monitoring accumulation of the compound of interest at the desired site in vivo by magnetic resonance imaging.

In certain embodiments, the present invention relates to any of the aforementioned methods, wherein monitoring the accumulation of the compound of interest at the desired site by magnetic resonance imaging comprises making a pixel density determination.

In certain embodiments, the present invention relates to any of the aforementioned methods, further comprising predicting efficacy of treatment based on a location of accumulation at the desired site, a rate of accumulation at the desired site, or both the location and the rate of accumulation at the desired site.

Another aspect of the invention relates to a method of targeting delivery of a compound of interest at a desired site in vivo, the method comprising: administering to a subject a composition comprising a liposome prepared by any of the aforementioned methods, provided that the liposome comprises a compound, wherein a non-physiological environmental condition is present at the desired site, and the composition is targeted to a desired location at the desired site in the subject, at a desired rate of accumulation at the desired site, or both a desired location and a desired rate of accumulation at the desired site by the presence of the non-physiological environmental condition.

In certain embodiments, the present invention relates to any of the aforementioned methods, wherein the liposome comprises DSPC/Cholesterol (55:45, mol:mol), DPPC-PEG₂₀₀₀, DPPC-DSPE-PEG₂₀₀₀ (95:5, mol:mol), or DPPC-MSPC-DSPE-PEG₂₀₀₀ (90:10:4, mol:mol).

In certain embodiments, the present invention relates to any of the aforementioned methods, wherein the non-physiological environmental condition is selected from the group consisting of hyperthermia, electromagnetic radiation, a chemical agent and non-physiological pH.

In certain embodiments, the present invention relates to any of the aforementioned methods, wherein the hyperthermia is provided by a natural process or a method selected from the group consisting of contacting a heated material with the desired site, applying RF energy to the desired site, applying microwave energy to the desired site, applying ultrasonic energy to the desired site and applying a laser beam to the desired site.

In certain embodiments, the present invention relates to any of the aforementioned methods, wherein the desired site is exposed to a non-physiological environmental condition before, after, or both before and after administering the composition.

In certain embodiments, the present invention relates to any of the aforementioned methods, comprising administering the composition in one or more partial doses before, after, or both before and after the desired site is exposed to a non-physiological environmental condition.

In certain embodiments, the present invention relates to any of the aforementioned methods, wherein the desired site is selected from the group consisting of a tumor, an embolism, an injury site, an ischemia, and at a tissue edema.

In certain embodiments, the present invention relates to any of the aforementioned methods, further comprising monitoring accumulation of the compound of interest at the desired site in vivo by magnetic resonance imaging.

In certain embodiments, the present invention relates to any of the aforementioned methods, wherein monitoring the accumulation of the compound of interest at the desired site by magnetic resonance imaging comprises making a pixel density determination.

In certain embodiments, the present invention relates to any of the aforementioned methods, further comprising predicting efficacy of treatment based on a location of accumulation at the desired site, a rate of accumulation at the desired site, or both location and rate of accumulation at the desired site.

### BRIEF DESCRIPTION OF THE FIGURES

FIG. 1 depicts the increase in fluorescence as doxorubicin is released from Gd-HP-DO3A-Dox-LTSL. A) Release of Dox as a function of temperature is shown as the sample is warmed from 20 to 55° C. at 1° C./min. Note that in this graph the effects of changes in temperature and time are coupled. B) In this assay of Dox release, temperature is kept relatively constant (see FIG. 6), and therefore time is the only independent variable. Percent release is calculated by assuming 100% release with Triton® X-100 and 0% release at 25° C. in a HEPES buffer.

FIG. 2 depicts the calibration of 1/T₁ versus concentration of ProHance® at 1.5T. Gd-HP-DO3A-Dox-LTSL solutions were heated in a water bath to release ProHance® and the drug. The resulting relaxivity (slope) values for lysed and intact Gd-HP-DO3A-Dox-LTSL were 4.01±0.10 and 1.95±0.05 mM⁻¹s⁻¹, respectively, and were significantly different (p<0.0001, F test). Relaxivity of ProHance® (4.05±0.14 mM⁻¹s⁻¹) was not significantly different from that of lysed Gd-HP-DO3A-Dox-LTSL (p=0.85, F test). R²>0.992 for all fitted data.

FIG. 3 depicts the release of Dox and ProHance® from Gd-HP-DO3A-Dox-LTSL. A) Percent release vs. time and fitted curves for Dox (—) and ProHance® (∘—) over 10 minutes. B) The first minute of release. Symbol size indicates temperature: 37° C. is smallest and 41.3° C. is largest. The percent release values are not significantly different between Dox and ProHance® (p>0.05, Dunn's multiple comparison), with an mean absolute differences between Dox and ProHance % Release of 2.8±1.5%, 6±4% and 3±2% for 37° C., 40° C. and 41.3° C. Each point represents the mean of 3 experiments±SEM.

FIG. 4 depicts the stability of Gd-HP-DO3A-Dox-LTSL. A) Release of doxorubicin immediately (dashed line) and 7 days after synthesis (solid line) of Gd-HP-DO3A-Dox-LTSL at 37, 40 and 41° C. B) Point-by-point difference between release curves at 37, 40 and 47° C. obtained 1 week apart (% release at day 0 to % release at day 7). Symbol size indicates temperature: 37° C. is smallest and 41° C. is largest. Median decrease in release of 0.13-1.9% was observed at all of the temperatures after 1 week of storage. This difference was significant at 37° C. (p<0.05, Dunn's multiple comparison test), but not at other temperatures (p>0.05).

FIG. 5 depicts an increase in magnetic resonance signal intensity due to release of ProHance® from Gd-HP-DO3A-Dox-LTSL caused by MRgHIFU in a silica-agarose gel phantom. During heating, the signal intensity of the region where the MRgHIFU beam is focused increases as ProHance is released. The signal intensity in a region that has been previously heated does not change, but stays high. An unheated region where ProHance® release does not occur shows a stable and low signal intensity.

FIG. 6 depicts the temperature during release assays at 37, 40 and 41.3° C. in which both doxorubicin and ProHance® release were quantified (FIG. 3). The initial decrease in temperature is due to the addition of concentrated liposomal solution to the pre-heated HEPES buffer.

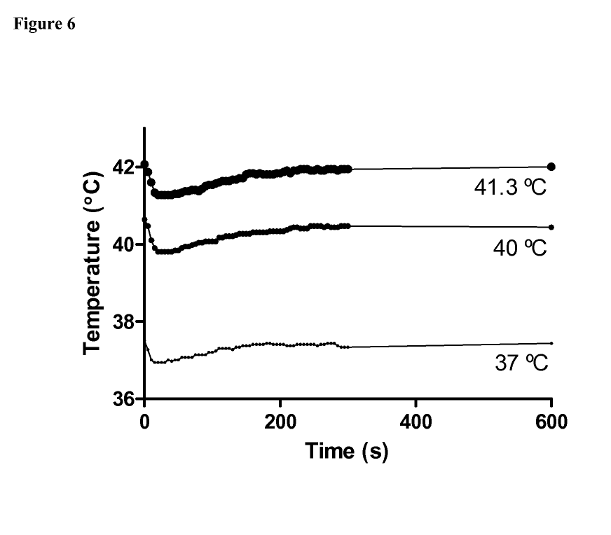

FIG. 7 depicts estimates of percent release of ProHance® at 37, 40 and 41.3° C. A) Release over 10 minutes. B) First minute of triggered release. The methods used to approximate % ProHance® release were T₁ measurements before (□) and after (Δ) passing aliquots through two size exclusion chromatography columns, as well as concentration measurements with ICP-AES (). Symbol size indicates temperature: 37° C. is smallest and 41.3° C. is largest. Maximum mean difference from ICP-AES measurements is 7±2 for □ and 9±4 for Δ. Percent release magnitudes were not different between ICP-AES and the other two methods of measurements (p>0.05, Dunn's multiple comparison). Each point represents the mean of 3 experiments±SEM.

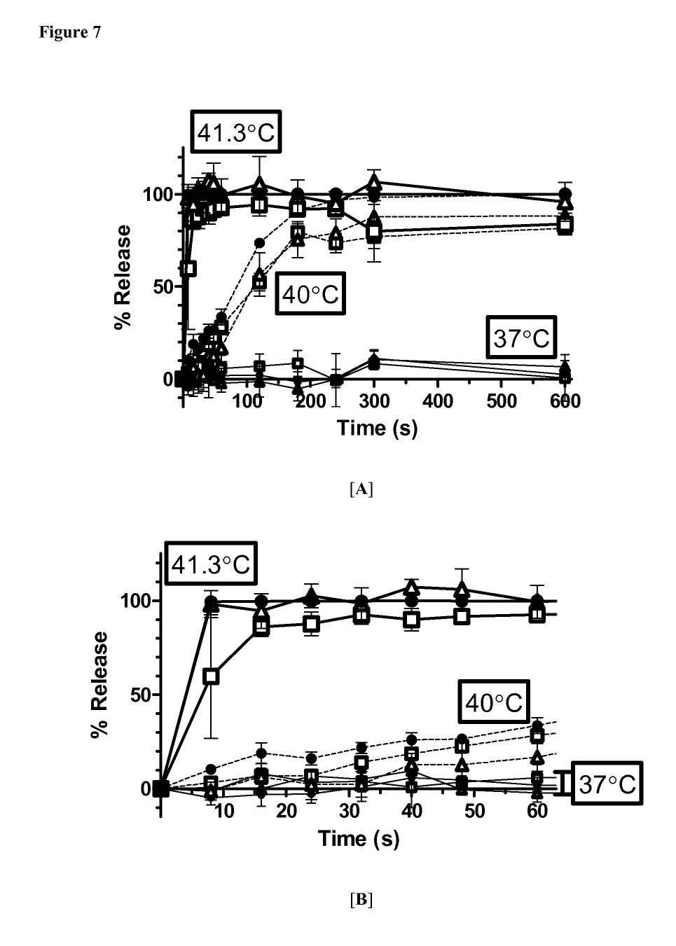

FIG. 8 depicts the relationship of particle size of the LTSLs comprising only Magnevist and the Magnevist concentration therein, prepared from solutions in A) citrate and B) water.

FIG. 9 depicts A) the relationship of particle size of LTSLs comprising only ProHance and the ProHance concentration therein, prepared from a citrate solution; and B) that the osmolarity of ProHance solution in citrate buffer may be maintained less than 600 mOsm with ProHance 400 mM and variable citrate concentration.

FIG. 10 depicts the Gd and Dox loading for LTSL comprising only Magnevist or ProHance.

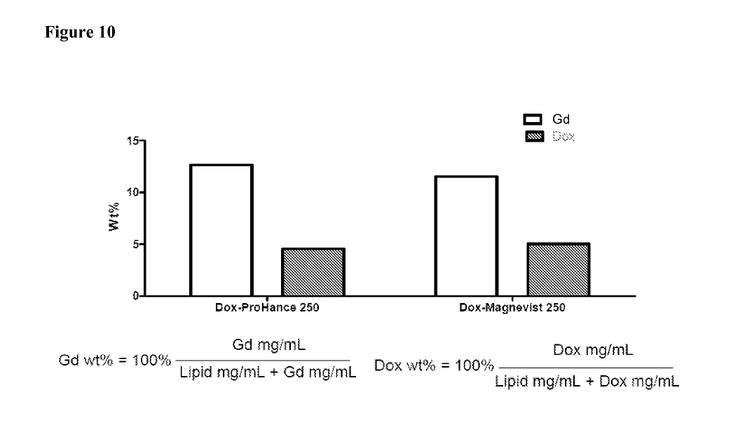

FIG. 11 depicts rapid ionophore-assisted Gd loading into LTSL comprising DTPA: A) 250 mM; and B) 150 mM.

FIG. 12 depicts how particle size increases with Magnevist concentration while relatively constant for various concentrations of ProHance.

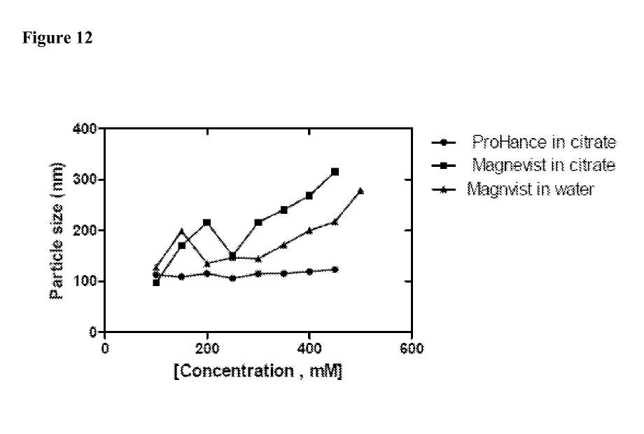

### DETAILED DESCRIPTION

The presently disclosed subject matter pertains in part to the preparation of liposome compositions for selected delivery of therapeutic agents, validated (qualitatively and quantitatively) by MRI, through the selective application of hyperthermia, and/or other non-physiological environmental conditions. Further, evaluation of drug distribution (qualitatively and quantitatively) can be used to predict treatment efficacy. In certain embodiments, the liposome compositions disclosed herein comprise a contrast agent (for example gadolinium-based compounds) and a therapeutically active compound of interest. In certain embodiments, these liposome compositions have higher contrast-agent loadings, and are more stable, than those previously reported.

In certain embodiments, the presently disclosed compositions can be used to validate temperature distribution in target tissues based on release profiles. In addition, the presently disclosed compositions can be used to target a drug to a desired site within a target tissue using selective application of a non-physiological environmental condition.

In some embodiments the presently disclosed subject matter provides methods of using the contrast agent-containing liposomes of the invention for quantitatively monitoring the accumulation of a compound in vivo by magnetic resonance imaging. Once this liposome composition is administered to a subject, the release from the liposome and accumulation of the compound can be monitored by magnetic resonance imaging, enabling the real time localization and distribution of the compound to a specific site to be imaged.

In certain embodiments, the liposomal compositions comprise envirosensitive liposomes (e.g., thermally-sensitive, pH-sensitive, chemosensitive, or radiation-sensitive liposomes), which can be prepared and employed, for example, in selective tissue targeting.

In addition, non-thermally sensitive liposomal compositions can be used to act as a blood pool agent, to identify tumors and assess uniformity of tissue perfusion. Further, the imageable liposomes disclosed herein can also be used for temperature measurements during hyperthermia treatment.

The presently disclosed subject matter can provide non-invasive measurement of drug distribution in real time. Qualitative as well as quantitative drug distribution can be assessed. Any desired therapeutic agent can be encapsulated into the presently disclosed liposomes. Selective delivery of a therapeutic drug can be provided, in some embodiments, by sensing, for example, inherent or imposed environmental variation within a tissue of interest. Local hyperthermia is a representative example of an environmental variation.

The presently disclosed subject matter can also provide the ability to monitor and/or predict in vivo concentration distributions, which can further provide for improvement in treatments. The presently disclosed subject matter can impact clinical treatment by providing individualized monitoring of tissue drug concentration distributions, allowing for modification of treatment variables (e.g., selective application of non-physiological environmental conditions) to improve the uniformity or selective targeting of drug delivery. The presently disclosed methods and compositions can provide individualized treatment, which in some applications can increase overall treatment efficacy.

### Definitions

Following long-standing patent law convention, the terms “a” and “an” mean “one or more” when used in this disclosure, including the claims.

As used herein, the term “about”, when referring to a value or to an amount of mass, weight, time, volume, concentration or percentage is meant to encompass variations of ±20% or ±10%, more preferably ±5%, even more preferably ±1%, and still more preferably ±0.1% from the specified amount, as such variations are appropriate to perform the disclosed method.

As used herein, the term “blood pool” means a localized volume of blood. A blood pool can arise from a normally occurring phenomenon, such as pooling of blood in a subject's heart. A blood pool can also arise from an unnatural vascular condition, such as an aneurysm. The blood in a blood pool can be circulating, prevented from circulating or circulating to some degree. Generally, then, a blood pool is a localized concentration of blood, and a blood pool can comprise any volume of blood.

As used herein, the term “detecting” means confirming the presence of a target entity (which can be a biological structure, such as a vascular blockage, vascular damage or an occlusion) by observing the occurrence of a detectable signal, such as a radiologic or spectroscopic signal or a feature of an image generated by magnetic resonance that will appear exclusively in the presence of the target entity.

As used herein, the term “envirosensitive liposome” means a liposome formulated using physiologically compatible constituents, such as, but not limited to, dipalmitoylphosphatidyl-choline and dipalmitoylphosphatidyl-glycerol phospholipids, which permit preparation of liposomes using art-recognized techniques that are formulated to lose structural integrity and release their contents under specific environmental conditions. The specific environmental conditions under which a particular envirosensitive liposome loses its structural integrity are variable and dependent upon the formulation of the particular liposome. Typically, the environmental conditions differ from normal physiological conditions. For example, thermosensitive liposomes can be formulated to release their contents at temperatures higher than normal mammalian body temperature. Alternatively, radiation-sensitive liposomes can be formulated to release their contents when they interact with electromagnetic radiation within a particular wavelength range, such as x-rays, or other ionizing radiation. While these examples are both categorized as envirosensitive liposomes, as the term is used herein, they are not necessarily structurally vulnerable to the same environmental conditions. For example, a thermosensitive liposome may not lose structural integrity when contacting x-rays, and vice versa for a radiation-sensitive liposome held at a particular temperature. However, envirosensitive liposomes having overlapping environmental sensitivities, for example thermal and pH sensitivities, can also be formulated, and are included within the term “envirosensitive liposome,” as used herein. One of skill in the art will readily recognize and be able to formulate without undue experimentation other types of envirosensitive liposomes, and these formulation are also encompassed by the term as used herein. Non-limiting examples of envirosensitive liposomes include thermosensitive liposomes, radiation-sensitive liposomes, pH-sensitive liposomes, acoustic (e.g. ultrasound)-sensitive liposomes, antigen-sensitive liposomes (e.g. liposomes having recognition molecules, for example, antibodies or antibody fragments (see, e.g., Sullivan & Huang, (1985) Biochim. Biophys. Acta 812(1): 116-126; Perlakv et al. (1996) Oncol. Res. 8(9): 363-369), incorporated into the membrane such that contact of the recognition molecule with its antigen results in loss of structural integrity of the liposome through, for example, a conformational change in the recognition molecule) and chemosensitive liposomes (e.g. liposomes sensitive to particular chemical agents). The present disclosure encompasses the use of envirosensitive liposomes in some embodiments that are less than about 400 nm in diameter, such as, for example liposomes having a diameter of about 200 nm, about 120 nm, about 100 nm, about 70 nm, about 60 nm, or about 50 nm in diameter to facilitate MRI visualization, handling, administration, unhindered progress through mammalian vasculature, and minimize side effects, e.g., interference with the mammalian blood clotting cascade.

As used herein, the term “hyperthermia” means the elevation of the temperature of a subject's body, or a part of a subject's body, compared to the basal temperature of the subject. Such elevation can be the result of a natural process (such as inflammation) or artificially induced for therapeutic or diagnostic purposes. In mammals, a basal body temperature is ordinarily maintained due to the thermoregulatory center in the anterior hypothalamus, which acts to balance heat production by body tissues with heat loss. “Hyperthermia” refers to the elevation of body temperature above the hypothalamic set point due to insufficient heat dissipation. In contrast to hyperthermia, “fever” refers to a systemic elevation of body temperature due to a change in the thermoregulatory center. The overall mean oral temperature for a healthy human aged 18-40 years is 36.8.±0.4° C. (98.2±0.7° F.). See, e.g., Harrison's Principles of Internal Medicine (Fauci et al., eds.) 14th Ed. McGraw-Hill, New York, p. 84 (1998).

As used herein, the term “inner transition elements” means those elements known as lanthanide (or rare earth) and actinide elements. Inner transition elements are also known as f-block transition elements.

As used herein, the term “liposome” means a generally spherical cluster or aggregate of amphipathic compounds, including lipid compounds, typically in the form of one or more concentric layers, for example, bilayers.

As used herein, the term “radiation-sensitive liposome” means a liposome formulated using physiologically compatible constituents, such as, but not limited to, dipalmitoylphosphatidyl-choline and dipalmitoylphosphatidyl-glycerol phospholipids, which permit preparation of liposomes using art-recognized techniques that are formulated to lose structural integrity and release their contents when interacting with electromagnetic radiation having a specific wavelength range. The specific wavelength range under which a particular radiation-sensitive liposome loses its structural integrity is variable and dependent upon the formulation of the particular liposome. For the purposes of example but not limitation, a liposome can be formulated to lose structural integrity and release its contents when interacting with x-rays, that is electromagnetic radiation having a wavelength in the range of about 1×10⁻¹¹m to about 1×10⁻⁸ m, but not when interacting with radiation having a greater or lesser wavelength. In other embodiments, the wavelength sensitivity may include a different range, or encompass x-rays in a broader range, such as, for example broad sensitivity to all ionizing radiation. The present disclosure encompasses the use of radiation-sensitive liposomes that are less than about 400 nm in diameter, such as, for example liposomes having a diameter of about 200 nm, about 120 nm, about 100 nm, about 70 nm, about 60 nm, or about 50 nm in diameter to facilitate MRI visualization, handling, administration, unhindered progress through mammalian vasculature, and minimize side effects, e.g., interference with the mammalian blood clotting cascade.

As used herein, the term “relaxivity” means the slope of the line drawn between points on a plot of 1/T₁ or 1/T₂ against contrast agent concentration.

As used herein, the term “subject” means any organism. The term need not refer exclusively to a human being, one example of a subject, but can also refer to animals such as mice, rats, dogs, poultry, livestock and even tissue cultures. The compositions and methods disclosed herein are particularly useful in the treatment and diagnosis of warm-blooded vertebrates.

As used herein, the term “T₁” means longitudinal relaxation time, and is also known as the spin lattice relaxation time (1/Ti is a rate constant, R₁, the spin-lattice relaxation rate constant).

As used herein, the term “T₂” means transverse relaxation time, which arises, in part, from spin-spin relaxation mechanisms. 1/T₂ is also a rate constant, R₂, the spin-spin relaxation rate constant.

As used herein, the term “non-sensitive liposome” means a liposome formulated using physiologically compatible constituents, such as dipalmitoylphosphatidyl-choline and dipalmitoylphosphatidyl-glycerol phospholipidsand cholesterol, that permits preparation of liposomes using art-recognized techniques that do not release their contents as a result of specific environmental stimulation, such as hyperthermic conditions, pH variance, or interaction with electromagnetic radiation. For example, in contrast to thermosensitive liposomes, non-sensitive liposomes (in this case also referred to as non-thermally sensitive liposomes) do not release their contents due to hyperthermic stimulation, such as at temperatures less than about 15 degrees higher than basal mammalian body temperature, i.e., above about 37° C. The present disclosure encompasses the use of non-sensitive liposomes that are less than about 400 nm in diameter, such as, for example liposomes having a diameter of about 200 nm, about 120 nm or about 100 nm in diameter to facilitate handling, administration, unhindered progress through mammalian vasculature, ability to target damaged or malformed vasculature, and minimize side effects, e.g., interference with the mammalian blood clotting cascade.

As used herein, the term “thermosensitive liposome” means a liposome formulated using physiologically compatible constituents, such as for example dipalmitoylphosphatidylcholine and dipalmitoylphosphatidylglycerol phospholipids, that permits preparation of liposomes using art-recognized techniques that release their contents at temperatures at least about 3 degrees higher than about 37° C. (normal mammalian body temperature). Upon exposure to temperatures at least about 3° C. above basal mammalian body temperature, release of liposome contents occurs by leakage or seepage or by actual lysis (complete or incomplete) of the liposomes. The present disclosure encompasses the use of thermosensitive liposomes that are less than about 400 nm in diameter, such as, for example liposomes having a diameter of about 200 nm, about 120 nm, about 100 nm, about 70 nm, about 60 nm, or about 50 nm in diameter to facilitate MRI visualization, handling, administration, unhindered progress through mammalian vasculature, and minimize side effects, e.g., interference with the mammalian blood clotting cascade.

As used herein, “liposome-forming lipid” is any lipid that is capable of forming liposomes. Typically, the “liposome-forming lipid” is a lipid that can form lipid bilayers. Examples of liposome-forming lipids include phospholipids, glycolipids and sphingolipids. The phospholipids that are liposome-forming include phosphatidylcholine, phosphatidylserine, phosphatidylinositol, phosphatidylglycerol, diphosphatidylglycerol and N-acyl phospatidylethanolamine. Examples of liposome-forming phospholipids include phospholipids selected from the group consisting of dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine, 1-oleoyl-2-palmitoyl-sn-glycero-3-phosphocholine, 1,2-dioleoyl-sn-glycero-3-[phospho-rac-(1-glycerol)], 1,2-dipalmitoyl-sn-glycero-3-[phospho-rac-(1-glycerol)], 1,2-distearoyl-sn-glycero-3-[phospho-rac-(1-glycerol)], 1,2-dimyristoyl-sn-glycero-3-[phospho-rac-(1-glycerol)], 1-palmitoyl-2-oleoyl-sn-glycero-3-[phospho-rac-(1-glycerol)], 1-oleoyl-2-palmitoyl-sn-glycero-3-[phospho-rac-(1-glycerol)], N-decanoyl phosphatidylethanolamine, N-dodecanoyl phosphatidylethanolamine and N-tetradecanoyl phosphatidylethanolamine.

In certain embodiments, the liposome-forming lipids include phosphatidylcholine, e.g., dioleoyl phosphatidylcholine, dipalmitoyl phosphatidylcholine, distearoyl phosphatidylcholine, dimyristoyl phosphatidylcholine, 1-palmitoyl-2-oleoyl-sn-glycero-3-phosphocholine and 2-palmitoyl-1-oleoyl-sn-glycero-3-phosphocholine, or N-acyl phosphatidylethanolamine, e.g., 1,2-dioleoyl-sn-glycero-N-decanoyl-3-phosphoethanolamine, 1,2-dioleoyl-sn-glycero-N-dodecanoyl-3-phosphoethanolamine, 1,2-dioleoyl-sn-glycero-N-tetradecanoyl-3-phosphoethanolamine, 1,2-dipalmitoyl-sn-glycero-N-decanoyl-3-phosphoethanolamine, 1,2-dipalmitoyl-sn-glycero-N-dodecanoyl-3-phosphoethanolamine, 1,2-dipalmitoyl-sn-glycero-N-tetradecanoyl-3-phosphoethanolamine, 1-oleoyl-2-palmitoyl-sn-glycero-N-decanoyl-3-phosphoethanolamine, 1-oleoyl-2-palmitoyl-sn-glycero-N-dodecanoyl-3-phosphoethanolamine, 1-oleoyl-2-palmitoyl-sn-glycero-N-tetradecanoyl-3-phosphoethanolamine, 1-palmitoyl-2-oleoyl-sn-glycero-N-decanoyl-3-phosphoethanolamine, 1-palmitoyl-2-oleoyl-sn-glycero-N-dodecanoyl-3-phosphoethanolamine, and 1-palmitoyl-2-oleoyl-sn-glycero-N-tetradecanoyl-3-phosphoethanolamine.

Certain embodiments of the preparatory methods of the present invention use one, or a combination (at any ratio), of the following lipids: phosphatidylcholines, phosphatidylglycerols, phosphatidylserines, phosphatidylethanolamines, phosphatidylinositols, headgroup modified phospholipids, headgroup modified phosphatidylethanolamines, lyso-phospholipids, phosphocholines (ether linked lipids), phosphoglycerols (ether linked lipids), phosphoserines (ether linked lipids), phosphoethanolamines (ether linked lipids), sphingomyelins, sterols, such as cholesterol hemisuccinate, tocopherol hemisuccinate, ceramides, cationic lipids, monoacyl glycerol, diacyl glycerol, triacyl glycerol, fatty acids, fatty acid methyl esters, single-chain nonionic lipids, glycolipids, lipid-peptide conjugates and lipid-polymer conjugates.

As used herein, the term “transition elements” means those elements found in columns IIIB, IVB, VB, VIIB, VIIIB, IB and IIB of the Periodic Table of Elements. Transition elements are also known as d-block elements.

As used herein, “Gd-DTPA” refers to the gadolinium complex of diethylene triamine pentaacetic acid, as shown below:

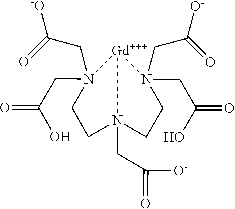

As used herein, “Magnevist®” or “gadopentetate dimeglumine” refers to 1-deoxy-1-(methylamino)-D-glucitol dihydrogen [N,N-bis[2-[bis(carboxymethyl)amino]ethyl]-glycinato-(5⁻-)-]gadolinate(2⁻) (2:1), as shown below:

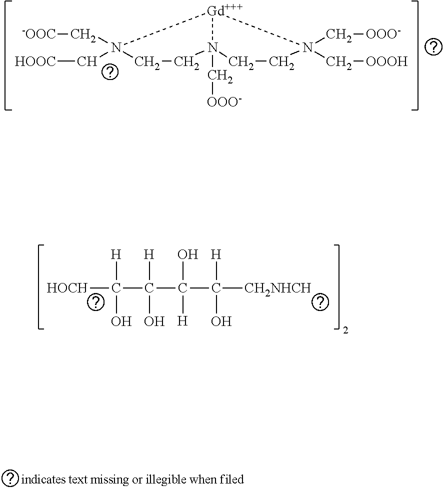

As used herein, “ProHance®,” “gadoteridol” and “Gd-HP-DO3A” refers to the gadolinium complex of 10-(2-hydroxy-propyl)-1,4,7,10-tetraazacyclododecane-1,4,7-triacetic acid, as shown below:

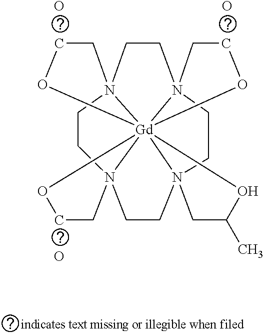

The following are some of the abbreviations used herein: AP, alkaline phosphatase; CT, computerized tomography; Dox, doxorubicin; ESR, electron spin resonance; HEPES, N-2-Hydroxyethylpiperazine-N′-2 ethanesulfonic acid; HT, hyperthermia; LTSL, lysolipid-based temperature sensitive (thermosensitive) liposomes; MLV, multilamellar vesicle; MR, magnetic resonance; MRI, magnetic resonance imaging; MUGA, multiple-gated arteriography; NMR, nuclear magnetic resonance; NTSL, non-temperature sensitive (non-thermally sensitive) liposomes; PET, positron emission tomography; RES, reticuloendothelial system; RF, radio frequency; SNR, signal-to-noise ratio; TCA, trichloroacetic acid; Te, echo time; TI, time of inversion; Tₘ, melting (phase transition) temperature; Tᵣ, repetition time; T₁, spin lattice relaxation time; and T₂, spin-spin relaxation time.

### General Considerations

One aspect of the present invention involves magnetic resonance (“MR”) -based techniques e.g., magnetic resonance imaging techniques. The magnetic resonance imaging techniques employed herein are known and are described, for example, in Kean & Smith, (1986) Magnetic Resonance Imaging: Principles and Applications. Williams and Wilkins, Baltimore, Md. U.S.A. Representative MR techniques include, but are not limited to, magnetic resonance imaging (“MRI”), nuclear magnetic resonance (“NMR”) and electronic spin resonance (“ESR”).

Standard MR equipment, conditions and techniques can be used to generate images. Appropriate equipment, conditions and techniques can be determined in the course of experimental design. When in vivo MRI experiments are performed in the context disclosed herein, they can be performed on any suitable MRI instrument, such as a 1.5 Tesla or higher whole-body scanner. According to known principles, nuclei with appropriate spin, when placed in an applied magnetic field (B₀, expressed generally in units of Tesla) align in the direction of the field. In the case of protons, these nuclei precess at a frequency, f, of 42.6 MHz, at a field strength of 1 Tesla (T). At this frequency, an RF pulse of radiation will excite the nuclei and can be considered to tip the net magnetization out of the field direction, the extent of this rotation being determined by the pulse energy (which can be obtained by integrating a time x amplitude curve). After the RF pulse, the nuclei “relax” or return to equilibrium with the magnetic field, emitting radiation at the resonant frequency. The decay of the emitted radiation is characterized by two relaxation times, i.e., T-i, the spin-lattice relaxation time or longitudinal relaxation time, that is, the time taken by the nuclei to return to equilibrium along the direction of the externally applied magnetic field, and T₂, the spin-spin relaxation time associated with the dephasing of the initially coherent precession of individual proton spins. These relaxation times have been established for various fluids, organs and tissues in different species of mammals.

MRI is a diagnostic and research procedure that uses a large, high-strength magnet and radio frequency signals to produce images. The most abundant molecular species in biological tissues is water. It is the quantum mechanical “spin” of the water proton nuclei that ultimately gives rise to the signal in standard imaging experiments. Other nuclei can be employed in MRI applications, however low signal-to-noise (S/N) ratios are a consideration in these applications. In an MRI experiment, the sample to be imaged is placed in a strong static magnetic field (on the order of 1-12 Tesla) and the spins are excited with a pulse of radio frequency (“RF”) radiation to produce a net magnetization in the sample. Various magnetic field gradients and other RF pulses then act on the spins to code spatial information into the recorded signals. The basic MRI experiment can be described, in one frame of reference, as follows. Pre-RF pulse spins can be thought of as collectively aligned along the Z-axis of a Cartesian coordinate system; application of one or a sequence of RF pulses “tip” the spins into the X-Y plane, from which position they will spontaneously relax back to the Z-axis. The relaxation of the spins is recorded as a function of time. Using this basic experiment, MRI is able to generate structural information in three dimensions in a relatively short period of time.

By applying magnetic field gradients so that the magnitude of the magnetic field varies with location inside the subject-receiving space characteristics of the magnetic resonance signals from different locations within the region, such as the frequency and phase of the signals, can be made to vary in a predictable manner depending upon position within the region. Thus, the magnetic resonance signals are “spatially encoded” so that it is possible to distinguish between signals from different parts of the region. After repeating this procedure with various different gradients, it is possible to derive a map showing the intensity or other characteristics of the magnetic resonance signals versus position within the excited region. Because these characteristics vary with concentration of different chemical substances and other characteristics of the tissue within the subject's body, different tissues provide different magnetic resonance signal characteristics. When the map of the magnetic resonance signal characteristics is displayed in a visual format, such as on screen or on a printed image, the map forms a visible picture of structures within the patient's body.

Two characteristic relaxation times are implicated in magnetic relaxation, the basis for MRI. T₁ is defined as the longitudinal relaxation time, and is also known as the spin lattice relaxation time (1/T₁ is a rate constant, R₁, the spin-lattice relaxation rate constant). T₂ is known as the transverse relaxation time, or spin-spin relaxation mechanism, which is one of several contributions to T₂ (1/T₂ is also a rate constant, R₂, the spin-spin relaxation rate constant). T₁and T₂ have inverse and reciprocal effects on image intensity, with image intensity increasing either by shortening the T₁ or lengthening the T₂.

In another aspect, the presently disclosed subject matter involves the use of the technique commonly referred to as “hyperthermia”. Hyperthermia, generally, is a technique for locally heating a site of interest to a temperature above normal body temperature. Hyperthermia is an established technique and forms the basis of several therapeutic regimens. For example, typical localized-hyperthermia temperatures required for therapeutic treatment of cancer are in the 42.5-45° C. range, which is maintained for approximately 30 to 60 minutes. Healthy tissue, however, should be kept at temperatures below 42.5° C. during the treatment. For targeted chemotherapy drug delivery, temperatures in the range of about 40 to 45° C. have been demonstrated to be effective on tumors. The presently disclosed subject matter, however, provides methods for using thermosensitive liposomes that can destabilize and release their contents at temperatures above basal temperature and below 42.5° C., thereby avoiding damage to healthy tissue.

In another aspect of the presently disclosed subject matter, a composition (e.g., an envirosensitive or non-sensitive liposome composition) can be introduced into a biological structure disposed in a subject. The mode of administration of a composition to a sample or subject can determine the sites and/or cells in the organism to which an agent will be delivered. The compositions can be administered in admixture with a pharmaceutical diluent (e.g., a buffer) selected with regard to the intended route of administration and standard pharmaceutical practice. The compositions can be injected into a subject parenterally, for example, intra-arterially or intravenously. For parenteral administration, a preparation can be used, e.g., in the form of a sterile, aqueous solution; such a solution can contain other solutes, including, but not limited to, salts or glucose in quantities that will make the solution isotonic. In another aspect, a composition can be injected directly into a tumor. In this aspect, the preparation will be injected in accordance with the above guidelines.

When a composition is administered to humans, the supervising physician or clinician will ultimately determine the appropriate dosage for a given human subject, and this can be expected to vary according to the weight, age and response of the subject as well as the nature of the subject's condition.

### Contrast Agents

Paramagnetic contrast agents serve to modulate tissue (or intrinsic) T₁ and/or T₂ values, and are typically designed with regard to a given metal nucleus, which is usually selected based on its effect on relaxation. The capacity to differentiate between regions or tissues that can be magnetically similar but histologically different is a major impetus for the preparation of these agents. Paramagnetic contrast agents provide additional image contrast, and thus enhanced images, of those areas where the contrast agent is localized. For example, contrast agents can be injected into the circulatory system and used to visualize vascular structures and abnormalities (see, e.g., U.S. Pat. No. 5,925,987; hereby incorporated by reference in its entirety), or even intracranially to visualize structures of the brain.

The measured relaxivity of the contrast agent is dominated by the selection of the metal atom. Paramagnetic metal ions, as a result of their unpaired electrons, act as potent relaxation enhancement agents. They decrease the T₁ relaxation times of nearby spins, exhibiting an r⁶ dependency, where r is the distance between the two nuclei. Some paramagnetic ions decrease the T₁ without causing substantial line broadening, for example copper(II) (“Cu(II)”), zinc(II) (“Zn(II)”), gadolinium(III) (“Gd(III)”) and manganese(II) (“Mn(II)”), while others induce drastic line broadening, for example, superparamagnetic iron oxide. The mechanism of T₁ relaxation is generally a through-space dipole-dipole interaction between the unpaired electrons of a metal atom with an unpaired electron (the paramagnet) and those water molecules not coordinated to the metal atom that are in fast exchange with water molecules in the metal's inner coordination sphere. When designing or selecting a liposome composition according to the present disclosure, an appropriate paramagnetic ion can be selected as a contrast agent. Any compound that affects the recovery of the magnetic moment of the water protons to the magnetic field, thereby reducing the Ti and T₂ relaxation times of an object of interest is suitable for use as a contrast agent with the methods and compounds disclosed herein. Some example metal ions suitable for use include, but are not limited to, the transition, lanthanide and actinide elements. For example, the metal ion is selected from the group consisting of Gd(III), Cu(II), Cr(III), Fe(II), Fe(III), Co(II), Er(II), Ni(II), Eu(III), Dy(III), Zn(II), Mg(II), Mo(III), Mo(VI), Li(I), Ta(V), Mn(II), and chelated forms thereof.

### Liposome Compositions

Drug delivery systems have been developed in which a drug-entrapping liposome composition is intravenously administered and delivered to a particular target site in the subject's body (see, e.g., Gregoriadis et al.; (1980) Receptor-mediated Targeting of Drugs, Plenum Press, New York, pp. 243-266). A requirement of such systems is that the liposome composition, after being intravenously or intra-arterially administered, should stably circulate along with blood in the subject's body for a longer period of time than provided by conventional systems.

Liposomes are generally not very stable in blood due to interactions between the liposomes' membrane component lipid and blood components such as lipoprotein. Also, intravenously and intra-arterially administered liposomes are sometimes recognized as a foreign substance by the reticuloendothelial system (RES) and are thus likely to be removed from the blood, due to the liposomes' physical morphology and biochemical properties. Significant efforts have been devoted to solving the problem of stabilizing liposomes in blood to avoid recognition by the RES, and thus, to enhance the liposomes' effective lifetime in the blood. For example, cholesterol has been added to liposome membrane compositions to increase blood liposome stability (Knight: (1981) Liposomes: From physical structure to therapeutic applications. Elsevier, North Holland, pp. 310-311). However, the effect thus obtained varies widely depending on the original membrane composition of the liposome (Senior et al. (1985) Biochim. Biophys. Acta 839:1-8). It has been reported that sialic acid-containing glycolipid, when administered as liposome, is distributed to the liver, a component of the RES (Surolia & Bachhawat (1977) Biochim. Biophys. Acta 497:760-765). It has also been reported that a drug was delivered into the brain after increasing the liposome's ability to pass through the blood brain barrier by functionalizing it with sulfatide, a glycolipid and a sulfo group (Naoi & Yaqi. (1984) Biochem. Int. 9:267-272). Recently, thermosensitive liposomes, liposomes that are stable at mammalian body temperature but become less stable at temperatures higher than mammalian body temperature, have been employed to encapsulate chemotherapeutic agents and to release these agents into heated tissue (see, e.g., U.S. Pat. No. 6,200,598 to Needham et al., incorporated in its entirety herein by reference). For example, successful targeted chemotherapy delivery to brain tumors in animals using thermosensitive liposomes has been demonstrated (Kakinuma et al., (1996) Int. J. Hyperther. 12(1):157-165). The results of this study indicated that when thermosensitive liposomes are employed as a drug carrier, significant chemotherapy drug levels were measured within brain tumors that were heated to the range of about 41 to 44° C. One formulation of a thermosensitive liposome is described in U.S. Pat. No. 5,094,854, incorporated in its entirety herein by reference.

In one aspect, the presently disclosed methods and compositions comprise envirosensitive or non-sensitive liposomes, e.g. thermosensitive and non-thermally sensitive liposome compositions. These liposomes can comprise virtually any particular combination of lipids, and can further comprise proteins, carbohydrates and other types of compounds as well. Generally, the same procedure can be employed for forming both envirosensitive and non-sensitive liposomes (e.g. thermosensitive and non-thermally sensitive liposomes), with the difference being in the lipid composition of the liposome.

Preparing Non-sensitive and Envirosensitive Liposomes. Envirosensitive or non-sensitive liposomes can be prepared utilizing techniques such as those employed in the art for conventional liposome preparation. Such conventional techniques are referred to, for example, in Published PCT International Application Serial No. WO 92/21017, incorporated in its entirety herein by reference, and by Papahadjopolous (Papahadiopolous. (1979) Ann. Rep. Med. Chem. 14:250-260) and include reverse evaporation, freeze-thaw, detergent dialysis, homogenization, sonication, microemulsification and spontaneous formation upon hydration of a dry lipid film. In one embodiment, a film of the lipid is deposited on a glass coverslip and then incubated in a sucrose solution for a predetermined time, such as 12 hours. A thin film of lipid is then deposited on the inside of a round bottom flask and then rehydrated at a temperature above its phase transition temperature (Tₘ). Then, the hydrated lipids are sonicated in order to form liposomes. Thermosensitive liposomes can be formed from a combination of lipids. Although almost any combination of lipids can be employed so long as the desired functional characteristic(s) is/are obtained, in one example, a. thermosensitive liposome comprises dipalmitoylphosphatidylcholine-polyethylene glycol (DPPC-PEG₂₀₀₀). In another example, a thermosensitive liposome comprises dipalmitoylphosphatidylcholine-distearoylphosphatidylethanolamine-polyethylene glycol (DPPC-DSPE-PEG₂₀₀₀) (95:5, mol:mol), and in yet another example, a thermosensitive liposome comprises polyenylphosphatidylcholine-MSPC-distearoylphosphatidylethanolamine-polyethylene glycol (DPPC-MSPC-DSPE-PEG₂₀₀₀) (90:10:4, mol:mol).

Other embodiments of envirosensitive liposomes include, but are not limited to, radiation-sensitive liposomes. The radiation-sensitive liposomes disclosed herein can be formed from a combination of lipids. Although almost any combination of lipids can be employed so long as the desired functional characteristic(s) is/are obtained, in one example, a radiation-sensitive liposome comprises dipalmitoylphosphatidylcholine (DPPC), distearoylphosphatidylcholine (DSPC), or diarachidoylphosphatidylcholine-polyethylene glycol (DAPC) in combination with PEG. In another example, cholesterol is added to one or more of these formulations. The addition of cholesterol reduces membrane fluidity and increases membrane integrity.

The radiation-sensitive formulations can further incorporate a radiation-sensitive lipid, which is selected based on the desired wavelength sensitivity. Thus, in one embodiment, liposomes can be made radiation-sensitive by the incorporation into the liposome wall radiation-sensitive lipids that undergo substantial alterations, such as isomerization, fragmentation or polymerization, upon interation with a particular wavelength range of electromagnetic radiation. For example, incorporation of polymerizable lipids such as 1,2-bis[10-(2′,4′-hexadienoyloxy)decanoyl]-sn-glycero-3-phosphocholine (bis-SorbPC), react directly with ultraviolet radiation in the presence of oxygen to form cross-linked polymer networks that significantly alter bilayer properties in the liposome wall, resulting in destabilization of the liposome and release of its contents (Bondurant et al. (2001) Biochimica et Biophysica Acta 1511:113-122). Spratt et al. have further shown that using a specific bis-SorbPC (bis-SorbPC_{19,19}) will increase reactivity to ultraviolet radiation by a magnitude of at least two orders (Spratt et al. (2003) Biochimica et Biophysica Acta 1611:35-43.

Sensitivity to other wavelength ranges of electromagnetic radiation can be achieved by incorporation of any of a variety of other sensitive lipids or even other non-lipid radiation-sensitive molecules. For example, visible light-sensitive liposomes can be formulated by incorporating dyes into the liposome walls. In one embodiment, ballasted cyanine dyes, such as distearoyl indocarbocyanine, in combination with polymerizable lipids, such as bis-SorbPC, are incorporated into liposome walls to produce light-sensitive liposomes that release their contents when contacted with visible light (green wavelength) (Mueller et al. (2000) Macromolecules 33:4799-4804).

For in vivo biological applications, it may be desirable to use radiation at wavelengths less absorbable by biomolecules than visible or ultraviolet light. Therefore, liposomes incorporating molecules providing radiation-sensitivity at other wavelengths, such as ionizing radiation (e.g. x-rays), or long wavelength radiation (e.g. near-infrared or infrared) can also be formulated similarly to the previously described radiation-sensitive liposomes. One of skill in the art would thus appreciate that radiation-sensitive liposomes providing sensitivity across the electromagnetic spectrum are encompassed by the methods and compositions described herein.

Also included in the methods and compositions described herein are pH-sensitive liposomes. As discussed for thermosensitive liposomes, almost any combination of lipids can be employed so long as the desired characteristics of pH-sensitivity at a particular pH range are obtained. Numerous pH-sensitive liposomes are known and described in the art (see, e.g., Litzinqer & Huang (1992) Biochim Biophys Acta 1113:201-227). In one example, dipalmitoyl phosphatidyl ethanolamine/palmitic acid provides a useful pH-sensitive liposome formulation (Lokling et al. (2001) Magnetic Resonance Imaging 19:731-738; and Lokling et al. (2003) Magnetic Resonance Imaging 21:531-540). Other formulations having similar properties are also encompassed by the present disclosure. Representative lipid compositions of non-sensitive liposomes include the thermosensitive liposome formulations disclosed herein, with the exception that the non-sensitive formulations often include cholesterol in varying amounts. For example, a non-sensitive liposome can comprise dipalmitoylphosphatidylcholine/cholesterol (DSPC/Cholesterol) (55:45, mol:mol). Envirosensitive as well as non-sensitive liposomes can be prepared by extrusion methods. Lipids, at certain ratios, such as those described above, can be dissolved in a chloroform-methanol mixture. The solvent can then be removed under a gentle stream of nitrogen gas and the lipid samples subsequently placed under a high vacuum for a time period of at least 4 hours to remove any residual solvent.

The dried samples can then be hydrated such that the final lipid concentration is, for example, about 100 mg/mL. In one embodiment, hydration can be achieved by contacting the dried samples with a hydrating buffer. Hydration of the lipid can be performed at a suitable temperature for a desired period of time, to generate multilamellar vesicles (MLVs).

Following hydration, MLVs can be extruded through stacked polycarbonate filters of, for example, 0.1 and 0.08 μm pore size at 55° C. using a water jacketed extrusion apparatus, such as an EXTRUDER™ apparatus (Northern Lipids Inc., Vancouver, British Columbia, Canada). Extrusion of the MLVs results in liposomes that are ready for loading.

Following preparation, the mean size distribution of a liposome preparation can be determined. For some preparation, a NICOMP Submicron Particle Sizer Model 270 (Pacific Scientific, Santa Barbara, Calif., United States of America) operating at 632.8 nm can be employed, although other methods and apparatuses can also be employed. Phospholipid can also be quantitated, for example by employing a known assay such as the Fiske and Subbarow phosphate assay (Fiske & Subbarow (1925) J. Biol. Chem. 2: 375-395)

In certain embodiments, once an envirosensitive or non-sensitive liposome is prepared, the liposome can be loaded with a contrast agent and/or a compound of interest, such as a drug.

Envirosensitive and non-sensitive liposomes can be loaded with a contrast agent and/or a compound of interest by employing any of a range of techniques known in the art. Various methods include osmotic loading, pH gradient-based loading and ionic gradient-based loading (Kulkarni et al. (1995) J. Microencapsul. 12(3): 229-46).

Contrast agent and/or a compound of interest can be loaded by generally following the method described by Abraham et al. (Abraham et al. (2002) Biochim. Biophys. Acta 1565:59-72).

Alternatively, in another embodiment, liposomes can be loaded using a pH gradient-based technique, such as described by Mayer et al. (Mayer et al. (1985) J. Biol. Chem. 260(2): 802-808). Generally, MLVs are produced by extrusion in the presence of K⁺ and then placed in a Na⁺ buffer to create a transmembrane Na⁺/K⁺ gradient with K⁺ concentrated within the vesicle and Na⁻ outside the vesicle. A contrast agent and/or a compound of interest in the buffer solution is accumulated at high concentrations within the liposome as a result of the transmembrane gradient. The rate of uptake of the contrast agent and/or compound of interest can be increased by the presence of an ionophore, such as, for example, valinomycin or Ionophore A23187. Uptake is also sensitive to pH with this system, and can be maximized based on the pK of the contrast agent and/or compound of interest.

This approach can be employed in the preparation of envirosensitive and non-sensitive liposomes. In certain embodiments, after liposomes are loaded, they can be stored in a suitable buffer solution and will be ready for use without further preparation.

### Design Criteria

In developing the liposomal compositions described herein, a set of key design criteria were identified, including stability (size and release profiles), image-ability (ability to report on content release in MRI) and ease of clinical translation. While these design criteria are discussed below, the specific liposomal compositions discussed are not intended to be limiting.

Stability. It was found that the stability of liposomal compositions comprising Gd-containing contrast agents could be optimized through the selection of the Gd-based agent identity and its concentration. For example, the buffer solution of 300 mM Gd-HP-DO3A in 100 mM citric acid used to prepare Gd-HP-DO3A-Dox-LTSL exhibited comparable osmolality to 300 mM citrate buffer commonly used to actively load Dox in liposmes.

Stability could be assayed by simple size measurements. For example, when the concentration of Gd-HP-DO3A was increased above 300 mM, particle size was larger immediately after preparation of the liposomes (data not shown), which indicated instability of the liposome. However, it was found that with 300 mM Gd-HP-DO3A, uniform particle size distribution and stable drug release were observed after storage of the liposome at 4° C. for a week. The increase in particle size as the concentration of Gd-HP-DO3A increases is attributed to the osmolarity increase of the liposome interior causing swelling. It is known that osmolarity is a colligative property and therefore depends on the total number of particles in solution. Due to its much higher osmolarity, it was found that encapsulation of Magnevist® (Magnevist-Dox-LTSL) resulted in unstable particle sizes above 0.15 mM Magnevist®. In comparison, Gd-HP-DO3A allowed a greater amount of contrast agent to be encapsulated.

Although the osmolarity of the loading solution was optimized, for Dox containing liposomal compositions, the true osmolarity of the solution inside the liposomes is not known, due to Dox transport through the liposomal membrane and the consumption of citrate which occurs during the Dox loading procedure. Therefore, in certain embodiments, the concentration citric acid was reduced to 100 mM to maintain a lower osmolarity. It was found that a 100 mM citric acid concentration was sufficient to load about 5 wt % Dox. In certain embodiments, the choice of contrast agent, and the need to optimize the osmotic balance across the liposomal membrane, is especially important for a liposome that is co-loaded with drug, since for such liposomes, drug dosing limits the amount of contrast agent that can be delivered.

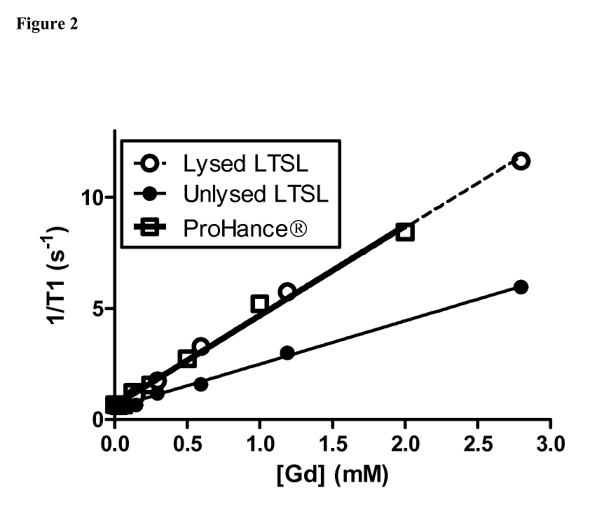

Image-ability. Image-ability of Gd-HP-DO3A-Dox-LTSL was optimized by selecting a concentration of contrast agent (i.e., Gd-HP-DO3A) that is capable of increasing magnetic resonance signals when a clinically relevant dose of Dox is administered in Gd-HP-DO3A-Dox-LTSL. For example, in certain embodiments, at the maximum tolerated dose of doxorubicin, the amount of Gd-HP-DO3A loaded into Gd-HP-DO3A-Dox-LTSL results in local concentrations of approximately 0.15 mM, which is known to increase magnetic resonance signal significantly at clinical magnet strength. In addition, for Gd-HP-DO3A-Dox-LTSL, the 66% increase in relaxivity after the contrast agent is released from the liposome (FIG. 2) points to the utility of this formulation in reporting on release of liposomal contents.

Clinical Translation. The clinical translation of Gd-HP-DO3A-Dox-LTSL might be eased by the choice of a liposomal formulation that is already in clinical trials along with a contrast agent that showed the lowest incidence of nephrogenic systemic toxicity (NSF) for Gd-based contrast agents. For example, a formulation that uses the similar materials and the same doxorubicin loading method (e.g., ThermoDox®) has been used in canine soft tissue sarcomas in a phase I trial (Hauck et al. (2006) Clin Cancer Res. 12(13):4004-10). A phase I trial of this formulation has been completed in humans for liver tumors in conjunction with radiofrequency ablation (Poon et al. (2009) Expert Opin Pharmacother. 10(2):333-43). The drug is now in a Phase III trial in this patient population (NCI PDQ 104-06-301, NCT00617981). It is also currently being tested in a phase I trial in patients with chest wall recurrences of breast cancer (NCI PDQ DUMC-6883-06-2R1, DUMC-06068, NCT00346229). However, as described in more detail below, a change in the loading procedure to add the contrast agent (e.g. Gd-HP-DO3A) and a reduction in the concentration of citric acid, results in improved contrast agent concentration and stability. Further, use of a contrast agent which is approved for use by the FDA and has not resulted in NSF (Port et al. (2008) Biometals. 21(4):469-90; and Kanal et al. (2007) American Journal of Roentgenology. 188(6):1447-74), may ease clinical translation and approval of the Gd-HP-DO3A-Dox-LTSL image-able liposomal formulation.

### Selected Applications

The presently disclosed methods and compositions can be employed in a variety of applications. Several of these applications are described in detail herein below. Additional applications of the presently disclosed methods and compositions will be apparent to those of ordinary skill in the art upon consideration of the present disclosure.

In the following applications, standard magnetic resonance imaging apparatus and methodology can be employed, as would be apparent to those of ordinary skill in the art after a review of the present disclosure.

Monitoring the Accumulation of a Compound of Interest at a Desired Site in vivo. In one application, a method of monitoring the accumulation of a compound of interest at a desired site in vivo by magnetic resonance imaging is disclosed. This application can be useful for tracking the delivery of a compound of interest to a site of interest, for example a tumor and for assuring that the compound of interest is delivered to the site in useful quantities.

In one embodiment, the method comprises increasing blood flow to a site of interest. As noted herein, a site of interest can be a tumor. In other examples, a site of interest can comprise a biological organ, such as the brain, liver, kidney or eye. In yet other examples, a site of interest can comprise a specific region or structure associated with the vasculature of a subject, or can even comprise the subject's vascular system in its entirety. After selecting a site of interest, blood flow is increased to the site of interest. Heating can be used as an effective approach for increasing the blood flow to the site of interest. The heat results in vasodilation at the desired site and a subsequent increase in blood flow to the site. Heating can be achieved by employing any of a variety of techniques. For example, a site can be heated by RF energy, by microwave energy, via application of ultrasonic energy or by conduction-based heating methods. When conduction-based heating methods are employed, one convenient method of heating is by contacting the site of interest with a catheter that is heated to a desired temperature, for example, with circulating water. When a site of interest is near an exposed surface of the subject (e.g., skin or eye), a laser can also be employed to heat the site.

A subject is then administered a non-sensitive liposome composition of the invention comprising (i) a contrast agent; (ii) a compound of interest; and (iii) a non-sensitive liposome encapsulating the contrast agent and the compound of interest.

Administration can be by an approach adapted to introduce the non-sensitive liposome composition into the bloodstream of the subject. For example, the administration can be by injection into an artery or vein. In one particular example, when a subject is a rat (for example, a Fisher 344 female strain rat), a liposome composition can be injected into the tail vein or femoral vein of the rat. Thus, administration can be, for example, via intravenous, intramuscular, intraperitoneal, intra-tumoral or subcutaneous intra-lesional injection.

A contrast agent can comprise any paramagnetic nucleus containing material, as disclosed herein above. Compounds comprising transition, lanthanide and actinide elements can also be employed. For example, a contrast agent can comprise an atom of gadolinium. A contrast agent can also comprise a chelate of the atom, such as for example gadoteridol, which is a gadolinium (III) chelate of 10-(2-hydroxypropyl)-1,4,7,10-tetraazacyclo-dodecane-1,4,7-triacetic acid (e.g., commercially-available ProHance®, Bracco Diagnostics, Inc., Princeton, N.J., U.S.A.).

A compound of interest can comprise any compound. For example, a compound of interest can comprise a pharmaceutically active compound, such as a chemotherapeutic compound (e.g. methotrexate, doxorubicin, cisplatinum, carboplatinum). A compound of interest can also comprise a compound suspected of being pharmaceutically active. In other cases, a compound of interest can generally comprise a compound known or suspected of modulating one or more biological processes. For example, a compound of interest can be a polypeptide or a polynucleotide.

The accumulation of the compound of interest at the site of interest may be monitored by magnetic resonance imaging. As the non-sensitive liposomes that have been administered to the subject circulate in the bloodstream of the subject, they will tend to accumulate at the site of heating. Over a given time interval, the presence of the liposomes at the heated site will increase. Thus, as time progresses, the presence of contrast agent at the heated site will concomitantly increase, since the contrast agent is encapsulated in the non-sensitive liposomes.

Magnetic resonance images of the heated site can be continuously and regularly generated. Methods of acquiring magnetic resonance images are established and can be employed to generate magnetic resonance images of a heated site. Since the contrast agent and the compound of interest are both encapsulated in the non-sensitive liposome, the accumulation of the contrast agent is directly proportional to the accumulation of the compound of interest.

It is noted that as the non-sensitive liposome compositions accumulate at the heated site, it is desired that they remain structurally coherent and the contents of the liposomes, namely a contrast agent and a compound of interest, remain inside the liposome. Thus, the accumulation of the compound of interest at a heated site can be monitored.

In certain embodiments, this technique can be performed in vivo, with the liposomes eventually being cleared by the renal and/or hepatic systems of the organism. It is possible to perform the method in vitro, on a tissue culture, for example, but most commonly the method will be performed in vivo on a subject.

In Vivo Method of Monitoring the Localization and Distribution of a Compound of Interest to a Desired Site in a Subject. Difficulties in delivering drugs to solid tumors in the human body have been documented. For example, abnormal vessels in tumors can restrict local blood flow in tumors and, hence, impede the delivery of drugs to the tumor. Abnormally elevated interstitial pressure within the tumor is also known to retard the passage of drug molecules from the blood stream into the tumor (Baxter & Jain (1989) Microvasc. Res. 37(1): 77-104; Baxter & Jain (1990) Microvasc. Res. 40(2):246-63; Baxter & Jain (1991) Microvasc. Res. 41(1):5-23; Baxter & Jain. (1991) Microvasc. Res. 41(2):252-72).

Effective cancer chemotherapy depends on delivery of therapeutic drugs to cancer cells at cytotoxic concentrations. Due to the inherent perfusion limitations that tumors present, delivery of drugs can be hindered. The ability to monitor and/or predict in vivo concentration distributions could improve treatment. Thus, in one aspect of the present disclosure, envirosensitive liposomes can be employed for in vivo monitoring of drug release and distribution from an envirosensitive liposome using MRI.

Additionally, the methods of loading envirosensitive and non-sensitive liposomes disclosed herein are applicable to a wider spectrum of compounds of interest (e.g., drugs) than was previously possible with pH loading methods, thereby broadening its applicability to other formulations.

In accordance with the present disclosure, an in vivo method of monitoring the distribution of a compound of interest to a desired site in an organism by magnetic resonance imaging is disclosed. In one embodiment, the method comprises increasing blood flow to a site of interest in a subject by, for example, applying heat. The site can be heated by external application of hot water, RF, ultrasound, or IR energy. Alternatively, interstitial application of energy can be obtained using the same physical methods. Further, in some instances, the site of interest can have an increased temperature due to an in vivo process (e.g., inflammation). The heat results in vasodilation at the desired site and an increase in blood flow to the site. Other methods of increasing vasodilatation and blood flow to the site are also acceptable for targeting the liposomes to a desired site. For example, direct mechanical massage or ultrasound treatment to the site can increase blood flow without the use of heat.

As described herein, a subject can be any living organism, for example, a human, mouse, rat, or rabbit, or a subject can be derived from a living organism and can comprise, for example, a tissue culture. A site of interest can comprise any biological structure. For example, a site of interest can comprise a tumor or an organ, such as a brain, liver, kidney, stomach, eye or lung.

A thermosensitive liposome composition can be administered to the subject. Again, the administration can be by convenient method, such as injection of the composition into a vein or artery of the subject.

An envirosensitive liposome composition of the present invention comprises a contrast agent; a compound of interest; and an envirosensitive liposome encapsulating the contrast agent and the compound of interest. The contrast agent can comprise any paramagnetic material. For example, contrast agent can comprise a paramagnetic material complexed with an organic material (e.g., a chelator) or an inorganic material (e.g., a sulfate moiety). A contrast agent can also comprise a chelate of the atom, such as for example gadoteridol.

A compound of interest can comprise any compound. Such a compound can comprise a chemotherapeutic agent, pharmaceutically active agent or an agent suspected to be of therapeutic value to the subject. Doxorubicin is employed as a non-limiting embodiment of such a compound in the Exemplification.

Additionally, one embodiment of the method comprises monitoring the localization and distribution of the compound of interest to the desired site by magnetic resonance imaging. The embodiment permits monitoring of both localization to the site of interest and distribution of the compound of interest to the site using magnetic resonance imaging. Distribution of the compound of interest refers to release of the compound from the liposome and dispersion of the compound at the site. The monitoring can be conveniently achieved by acquiring magnetic resonance images at any desired time point. Standard magnetic resonance techniques can be employed to generate such images.

In one embodiment, envirosensitive liposomes in the form of thermosensitive liposomes are used. The thermosensitive liposomes are stable at temperatures near mammalian body temperature, about 37° C. The temperature of the heated site will be several degrees above 37° C. As the thermosensitive liposomes travel to the heated site (e.g., through the circulatory system of the subject), they will accumulate at the heated site due, in part, to their size and release their contents due to their thermoinstability.

After exposure to heat for a period of time, the thermosensitive liposomes will become “leaky”. That is, the thermosensitive liposomes will lose a degree of structural integrity, allowing the contents of the liposomes, namely a contrast agent and a compound of interest, to be released from the thermosensitive liposome. The release of the contents of the thermosensitive liposome can be tracked by monitoring an increase in the presence of contrast agent at a range of points around a given structure. The association of contrast agent with a structure can be denoted by an increase in the pixel density and/or intensity of signal around the structure in a MR-generated image.

In a similar embodiment, envirosensitive liposomes in the form of radiation-sensitive liposomes are used. Like other envirosensitive liposomes, including the previously described thermosensitive liposomes, radiation-sensitive liposomes are stable under normal physiological conditions.

The radiation-sensitive liposomes also travel to and accumulate at the heated site due, in part, to their size. However, unlike thermosensitive liposomes, the radiation-sensitive liposomes will not be expected to release their contents when heated, unless the heat source is producing a wavelength of electromagnetic radiation within the range of sensitivity of the particular radiation-sensitive liposomes. Instead, a source of electromagnetic radiation emitting radiation at a wavelength within the range of sensitivity of the particular radiation-sensitive liposomes is directed at the site, which then interacts with susceptible lipids in the liposome wall. The sensitive lipids then either isomerize, fragment or polymerize, which then causes the liposomes to lose structural integrity and increase permeability, in some formulations, sufficiently to release their contents. Namely, the radiation-sensitive liposomes become permeable enough to at least allow exchange of water across the membrane. The membrane in some formulations will become sufficiently permeable to release the contained contrast agent and/or compound of interest. As already described, the release of contents can be tracked by monitoring the presence of contrast agent at a range of points around a given structure.

One of skill in the art will appreciate that other liposome disruption agents can be used, such as pH variance, depending on the disruption characteristics of the particular envirosensitive liposome formulated and the local environment deviation from normal tissue (e.g., from an in vivo process or applied externally).

In another aspect, drug release can also be quantified. One method of quantifying drug release generally involves employing a plot of concentration against 1/T₁ or 1/T₂ as a calibration curve. Continuing with this embodiment, before a given experiment is performed, the T₁ of a pixel is measured. This T₁ measurement can provide additional information, including proton density and base line noise. If all the imaging parameters (e.g., Tᵣ, PD, etc.) are kept constant, which is typical in a dynamic study, a change in signal intensity at a time point later than time t=0 is accompanied by a reduction in T₁. Such a change in T₁ is indicative of localization and distribution of a drug from an envirosensitive liposome composition. This reduction in T₁ can be converted to concentration using the plot of concentration against 1/T₁ as a standard curve for the corresponding compound. Thus, observed changes in T₁, which are associated with drug release, can be translated into a released drug concentration by indexing the observed T₁ with a given concentration on a plot of concentration against 1/T₁.

Method of Detecting an in vivo Blood Pool. Contrast agents with prolonged presence in the blood (i.e., good resistance to uptake by RES and a relatively low diffusivity into the tissue or extravascular locations) are recognized in the art as useful “blood pool agents” (see, e.g., U.S. Pat. No. 5,464,696, herein incorporated by reference in its entirety). Contrast agents exhibiting long biological half-lives are sometimes desirable for the blood pool agents if a researcher or clinician desires to produce meaningful analytical results and to eliminate repeated injections and the repeated use of a contrast agent. Several attempts to produce compositions suitable for use as blood pool agents have been made, including some for use with MRI (see, e.g., U.S. Pat. Nos. 5,833,948; 5,464,696; 6,010,681; 5,961,953; and 5,888,476, herein incorporated by reference in its/their entirety). Particularly, there has been an ongoing effort to develop contrast agents with long residence times in the blood circulation, that exhibit high relaxivity and can be completely eliminated from the system of a subject (i.e., agents that can be employed as “blood pool agents”).

Some efforts have focused on identifying and preparing paramagnetic substances encapsulated into liposome vesicles, immobilized in the liposome membrane, copolymerized with polyethylene glycol or grafted on a polymeric chain such as albumin, dextran or polylysine. Examples of such compositions include Gd-DTPA-albumin, Gd-DTPA-dextran or Gd-DTPA-polylysine complex molecules (see, e.g., Qqan et al. (1987) Invest. Radiol. 22:665; Wang et al. (1990) Radiology 175:483; Schumann-Giampieh et al. (1991) Invest. Radiol. 26:969; Vexler et al. (1994) Invest. Radiol. 29 supl. 2:S62; Dessler et al. (1994) Invest. Radiol. 29 supl. 2:S65; Meyer et al. (1994) Invest. Radiol. 29 supl. 2:S90; Shen et al. (1994) Invest. Radiol. 29 supl. 2:S217).

Notwithstanding, the half-life of contrast agents containing paramagnetic species bonded to macromolecules is often too short to be convenient for blood-pool imaging or have unexpected toxic side effects. In order to solve this difficulty, the use of suspensions of liposomal microvesicles containing encapsulated paramagnetic chelates as carriers of NMR contrast agents has been proposed. Use of liposomes for carriers has been proposed for relative biocompatibility and ease of preparation of liposomes and their suspensions. Encapsulation of known paramagnetic contrast agents into liposomes has been described (see, e.g., Unger et al. (1993) J. Mag. Res. Imag. 3:195-198).

These known compositions exhibit longer dwelling times in the blood than the water-soluble metal complexes; however, their residence times in the circulation are still not sufficient and some of these compounds have shown unacceptable levels of toxicity for blood-pool imaging. Longer residence times and lower immunogenicity have been reported by Bogdanov et al. (Boqdanov et al. (1993) Radiology 187:701) for Gd-DTPA-MPEG-polylysine complexes which consist of a methoxy(polyethylene glycol)-shielded macromolecular backbone (polylysine) bearing covalently attached Gd-DTPA. However, these prior art compositions do not offer the advantages of the compositions and methods disclosed herein.

Desirable properties of a blood pool agent include the ability to remain in a subject's bloodstream for protracted periods of time. For contrast agents administered into the systemic vasculature, as a general rule, low molecular weight hydrophilic molecules (e.g. molecular weight beneath about 5000 Da) distribute into the extracellular fluid (ECF) and are relatively rapidly excreted through the kidneys by glomerular filtration. Particulates, liposomes or lipophilic molecules tend to accumulate relatively rapidly in the liver. Thus, an effective blood pool agent would not be recognized by the RES, and would remaining in the bloodstream for an extended period, yet would still provide a magnetic relaxation response. The blood pool agents disclosed herein accomplish this goal.

The use of a blood pool agent can facilitate a wide range of measurements that can be of interest to researchers and clinicians. For example, one role that a blood pool agent can play is as an aid in the measurement of blood volumes and the blood perfusion of various organs, including the brain, using in vivo, non-invasive techniques.

Accordingly, in one aspect of the present disclosure, a method of detecting an in vivo blood pool is disclosed. In one embodiment of the method, a subject is administered a non-sensitive liposome composition. A suitable non-sensitive liposome composition can comprise a contrast agent and a non-sensitive liposome encapsulating the contrast agent. The administering can be carried out by any convenient method, although many times injection of the composition into a subject's vein or artery can be the most convenient approach to administering a composition.

After a non-sensitive liposome composition has been administered to a subject (which can be performed by employing a method disclosed herein or known to those of ordinary skill in the art), a magnetic resonance image of a site of interest can be generated. As noted throughout the present disclosure, a magnetic resonance image can be generated by any known method and can be generated on any available MRI apparatus, such as a 1.5T, 2T, 3T, 4T, or 7T whole-body clinical scanning instrument (e.g., MRI apparatus available from General Electric of Milwaukee, Wis., United States of America or from Siemens, Munich, Germany). One of skill in the art will appreciate, however, that the relaxivity of the liposomes is field strength dependent. As the field strength increases the relaxivity at a given temperature decreases. Although this can result in a reduced contrast for a given concentration, there is also an overall increase of the signal to noise from the higher field strength, such that sensitivity to the contrast agent should increase (i.e., signal to noise increases at a faster rate than the rate of relaxivity decrease as field strength is increased).

In one example of the method, a single image can be generated at a time point known or suspected to permit enough time for the envirosensitive liposome composition to circulate through the subject's blood stream to a site of interest. In another example of the method, a time course series of images of a site of interest can be acquired. Such a time course of images can be focused on a particular region of interest, such as the brain, or on a biological structure known or suspected to have a vascular irregularity. Continuing with the embodiment of the method, the presence of an in vivo blood pool can be detected by analyzing the magnetic resonance image. Such an analysis can comprise an evaluation of one or more MR images to identify the presence or absence of a blood pool at a particular site of interest. The presence of a blood pool is indicated, in a MR image, by the pixelation associated with a contrast agent. When images are black- and-white images, the contrast agent pixelation will show up as white pixelation.

As noted, the presence of a blood pool can be indicative of a vascular irregularity. A vascular irregularity can be, for example, a widening of a vascular structure. In one embodiment, a vascular irregularity can comprise an aneurysm. In another embodiment, a vascular irregularity can comprise an ischemic condition.

Ischemia/reperfusion injury is a significant source of morbidity and mortality in a number of clinical disorders, including myocardial infarction, cerebrovascular disease, and peripheral vascular disease. In addition, ischemia/reperfusion is relevant to the function of transplanted organs and to the recovery expedience following any cardiovascular surgery (see, e.g., Fan et al. (1999) J. MoI. Med. 77:577-596). Often times, ischemic conditions are not identified in a subject until after significant damage or death has resulted. Thus, the presently disclosed subject matter can be employed to monitor the formation, dissolution and properties of a blood pool, which can useful in the diagnosis and prevention of disorders related to vascular diseases and conditions.

Method of Generating a Heating Profile of a Site of Interest. In another aspect, a method of generating a heating profile of a site of interest is disclosed. The term “heating profile”, as it is used herein, encompasses the heating of a region of tissue surrounding a site of heating. A heating profile reflects the increase and/or decrease in heat as a function of distance from the site of heating or from the source of heat (e.g., a heated catheter or in vivo process, such as inflammation).

In one embodiment, the method comprises administering to a subject a thermosensitive liposome composition comprising: (i) a contrast agent and (ii) a thermosensitive liposome encapsulating the contrast agent and the compound of interest and having a melting temperature, Tₘ. Thermosensitive liposome compositions can be formed as described herein. The thermosensitive liposomes of such compositions will have a given melting temperature, which can be a function of the composition of the liposome. At temperatures below the Tₘ, the thermosensitive liposome retains its structural integrity; above the Tₘ, the thermosensitive liposome loses its structural integrity, allowing release of the liposome's contents. Representative contrast agents are described herein and can comprise, for example, Gd-chelates.

Continuing with the method, a site of interest in a subject is then heated. Various methods of heating can be employed in the method, such as heating via a catheter warmed by passing hot water through the catheter. Other heating methods are described herein. The release of the contrast agent from the thermosensitive liposome is then monitored using magnetic resonance imaging. The steps for acquiring such a magnetic resonance image are described herein. Standard MRI methodology can be employed in the acquisition of the image as disclosed herein and also will be known to those of ordinary skill in the art upon consideration of the present disclosure.

A heating profile of the site of interest can then be generated. In such a heating profile, the heating of an area to a temperature of at least Tₘ can optionally be indicated by release of contrast agent at a periphery of the area. Such a heating profile can reflect the distance from a site of heating (e.g., the radial distance) at which the Tₘ of the thermosensitive liposome is reached.

By way of example, a heated catheter can be employed to heat a tumor. The tumor tissue will be warmest near the site at which the catheter contacts the tumor, and will be cooler at points further away from the catheter. When the tumor tissue is homogeneous, this decrease in temperature as a function of distance from the catheter can reflect a linear or exponential decrease. At some distance from the catheter, the temperature of the tumor tissue will be equal to the Tₘ of a given thermosensitive liposome composition. When thermosensitive liposomes reach this distance (as disclosed herein, envirosensitive (e.g. thermosensitive), and non-sensitive, liposomes accumulate at a site of heating) they melt and release their contents (or merely accumulate if non-sensitive), namely a contrast agent. By evaluating MR images acquire as the thermosensitive liposomes approach the site of heating, the distance at which the Tₘ of the liposomes is reached can be determined. At the distance from the catheter that the tissue is heated to Tₘ, contrast agent release will be immediate and will resemble a burst release. Thus distances equal to or less than the Tₘ distance from the catheter can be identified, giving rise to a heating profile.

In another embodiment, two or more thermosensitive liposome compositions can be employed, for example, in succession. In this embodiment, the liposomes can have lipid compositions that impart different melting points. By administering several compositions, each with a different Tₘ, and compiling the results, a detailed heating profile, similar to a plot of different isotherms can be generated, which reflects the temperature of the tissue at various distances from the catheter or site of heating.

Methods of Predicting and Enhancing Efficacy of a Treatment. As disclosed herein, MRI techniques can be used to observe in vivo content release from liposomes that contain a contrast agent with or without a compound of interest. Specifically, local tissue concentrations of the compound of interest can be estimated from the shortening of MR T₁ relaxation times. For example, MRI can be used to measure temporal and spatial patterns of drug delivery in a rat fibrosarcoma model during treatment with liposomal compositions and hyperthermia administered with different schedules. Thereby, the pattern of drug delivery with envirosensitive liposomes can be controlled and monitored based on the perfusion pattern at the desired site and the temperature profile at the time of liposome administration. Thus, the use of different protocols of non-physiological condition exposures at a site of interest in conjunction with administration of the envirosensitive liposomes can permit compound of interest distribution to be controlled in real time, which is referred to herein as “drug dose painting.”

In view of the above discussion, in some embodiments of the presently disclosed subject matter a method of predicting efficacy of a treatment in a subject is provided. In some embodiments, the method comprises monitoring accumulation of a compound of interest at a desired site in vivo by magnetic resonance imaging and predicting efficacy of treatment based on accumulation of a compound of interest at the desired site.

In some embodiments, the method comprises administering to a subject a non-sensitive liposome composition and monitoring the accumulation of the compound of interest at the desired site by magnetic resonance imaging. In some embodiments, the non-sensitive liposome composition comprises a contrast agent, a compound of interest, and a non-sensitive liposome encapsulating the contrast agent and the compound of interest.

In other embodiments, the method comprises administering an envirosensitive liposome composition to a subject and monitoring the accumulation of the compound of interest at the desired site by magnetic resonance imaging (e.g., making a pixel density determination). In some embodiments, the envirosensitive liposome composition comprises a contrast agent, a compound of interest, and an envirosensitive liposome encapsulating the contrast agent and the compound of interest. In some embodiments, the method further comprises exposing the envirosensitive liposome at the desired site to a non-physiological environmental condition, which can be selected based on the nature of the envirosensitive liposome utilized. For example, the envirosensitive liposome can be thermosensitive, and the non-physiological environmental condition can be hyperthermia. Additional exemplary envirosensitive liposomes suitable for use include pH-sensitive liposomes, chemosensitive liposomes and radiation-sensitive liposomes.

In some embodiments, predicting efficacy comprises predicting efficacy of treatment based on a location of accumulation at the desired site, a rate of accumulation at the desired site, or both location and rate of accumulation at the desired site. The location of accumulation at the desired site can be a particular targeted region within or proximate to the desired site. For example, in a tumor, it may be desirable to target the periphery or outer regions of the tumor where vasculature feeds the tumor. The periphery of a tumor can be targeted for delivery of a therapeutic agent by exposing and equalizing the tumor to a non-physiological environmental condition (e.g., hyperthermia when utilizing thermosensitive liposomes), prior to administration of the envirosensitive liposome containing the therapeutic compound such that the liposome is induced to release the therapeutic compound at the periphery of the tumor, when it first encounters the non-physiological environmental condition. In contrast, if distribution to a central region of a tumor is desired, the envirosensitive liposomes can be administered prior to treatment with the non-physiological environmental condition, such that the liposomes accumulate within the central region of the tumor first and are then stimulated to release the therapeutic agent after treatment with the non-physiological environmental condition. In addition, uniform distribution can be accomplished by a combination of the above techniques, that is, a portion of the liposomes are administered prior to non-physiological environmental condition treatment and a portion administered after treatment. Rates of accumulation of the compound of interest at the desired site can also be predicted and manipulated based on the timing and intensity of non-physiological environmental condition treatment. For example, if rapid release of the compound of interest from the envirosensitive liposomes is desired, the treatment can be initiated prior to administration of the liposomes at the site of interest.

Related to methods of predicting efficacy of treatment, in some embodiments of the invention, a method of enhancing efficacy of a treatment at a desired site in a subject is provided. In some embodiments, the method comprises administering to the subject a composition comprising a compound of interest and targeting the composition to a desired location at a desired site in the subject, at a desired rate of accumulation at the desired site, or both a desired location and desired rate of accumulation at the desired site, to thereby enhance efficacy of treatment provided by the compound of interest. In some embodiments, composition is targeted to the desired location and/or at the desired rate by exposing the desired site to a non-physiological environmental condition, such as for example hyperthermia, electromagnetic radiation, a chemical agent and non-physiological pH. In some embodiments, the desired site is exposed to a non-physiological environmental condition before, after, or both before and after administering the composition to target the composition. Further, in some embodiments, targeting the composition comprises administering the composition in one or more partial doses before and/or after exposing the desired site to a non-physiological environmental condition.

In some embodiments, the method further comprises monitoring accumulation of the compound of interest at the desired site in vivo by magnetic resonance imaging. In some embodiments, monitoring the accumulation of the compound of interest at the desired site by magnetic resonance imaging comprises making a pixel density determination.

Additionally, in some embodiments of the presently disclosed subject matter, a method of targeting delivery of a compound of interest at a desired site in vivo is provided. In some embodiments, the method comprises administering to a subject a composition comprising a compound of interest and exposing the desired site to a non-physiological environmental condition to thereby target the composition to a desired location at the desired site in the subject, at a desired rate of accumulation at the desired site, or both the desired location and the desired rate of accumulation at the desired site. In some embodiments, the desired site is exposed to a non-physiological environmental condition before, after, or both before and after administering the composition to target the composition. Further, in some embodiments, targeting the composition comprises administering the composition in one or more partial doses before and/or after exposing the desired site to a non-physiological environmental condition.

In the disclosed methods, a compound of interest can comprise any compound. Such a compound can comprise a chemotherapeutic agent, pharmaceutically active agent or an agent suspected to be of therapeutic value to the subject. Doxorubicin is employed as a non-limiting embodiment of such a compound in the Exemplification. Further, the contrast agent can comprise any paramagnetic material, for example Gd, or any compound comprising, for example, a transition element or an inner block element. A contrast agent can comprise a paramagnetic material complexed with an organic material (e.g., a chelator, such as for example gadoteridol) or an inorganic material (e.g., a sulfate moiety). Exemplary contrast agents can comprise one or more elements selected from the group consisting of Gd, Cu, Cr, Fe, Co, Er, Ni, Eu, Dy, Zn, Mg, Mo, Li, Ta, and Mn.

In some embodiments of the disclosed methods, the non-sensitive liposome can comprise DSPC/Cholesterol (55:45, mol:mol). Further, the thermosensitive liposome can comprise a formulation selected from the group consisting of DPPC-PEG₂₀₀₀, DPPC-DSPE-PEG₂₀₀₀ (95:5, mol:mol) and DPPC-MSPC-DSPE-PEG₂₀₀₀ (90:10:4, mol:mol).

Further, in some embodiments of disclosed methods the desired site is selected from the group consisting of a tumor, an embolism, an injury site, an ischemia, and at a tissue edema.

### EXEMPLIFICATION

The invention now being generally described, it will be more readily understood by reference to the following examples, which are included merely for purposes of illustration of certain aspects and embodiments of the present invention, and are not intended to limit the invention.

In view of the many possible embodiments to which the principles of our invention may be applied, it should be recognized that illustrated embodiments are only examples of the invention and should not be considered a limitation on the scope of the invention.

Methods to load gadolinium inside a low temperature sensitive liposome (LTSL) containing Doxorubicin are described below. The first method (Method I), known as a passive loading method, uses a commercially available FDA approved contrast agent, such as Magnevist® or ProHance®, which is first lyophilized then suspended in a citrate buffer to later load doxorubicin (either active or passive loading). The second method (Method II), known as an active method, uses an ionophore to transfer gadolinium across the lipid bylayer of a liposomal composition comprising Dox. The gadolinium is then chelated by, for example, DTPA, thereby forming a compound similar to Magnevist® inside the liposome. Method II requires less gadolinium than Method I to achieve near equivalent gadolinium loading.

The following are some of the materials which were used in the experiments described below Doxorubicin (“Dox”); Magnivest®; ProHance® (“Gd-HP-DO3A”); Citrate buffer (300 mM, pH=4); HEPES buffer (pH 7.4, 10 mM, 280 mOsm); 1,2-dipalmitoyl-sn-glycero-3-phosphocholine (DPPC, Tₘ=41.5-41.9° C.); Monostearoyl-2-hydroxy-sn-glycero-3-phosphocholine (MSPC); 1,2-Distearoyl-sn-glycero-3-Phosphoethanolamine-N-[Amino(Polyethylene Glycol)2000] (“DSPE-PEG₂₀₀₀” or “DSPE-PEG”); Triton X-100; Hydrating buffer 1 (150 mM citrate, 45 mM diethylene triamine pentaacetic acid (DTPA), pH 4.0, 338 mOsm); Hydrating buffer 2 (150 mM citrate, 150 mM DTPA, pH 4.0, 612 mOsm); Hydrating buffer 3 (150 mM citrate, 250 mM DTPA, pH 4.0, 823mOsm); Arginine; External buffer (250 mM sucrose, 20 mM HEPES, pH 7.4, 301 mOsm); GdCl₃; A23187 ionophore; and Deionized water.

### Preparation of Magnivest-LTSL-Dox (Method I)

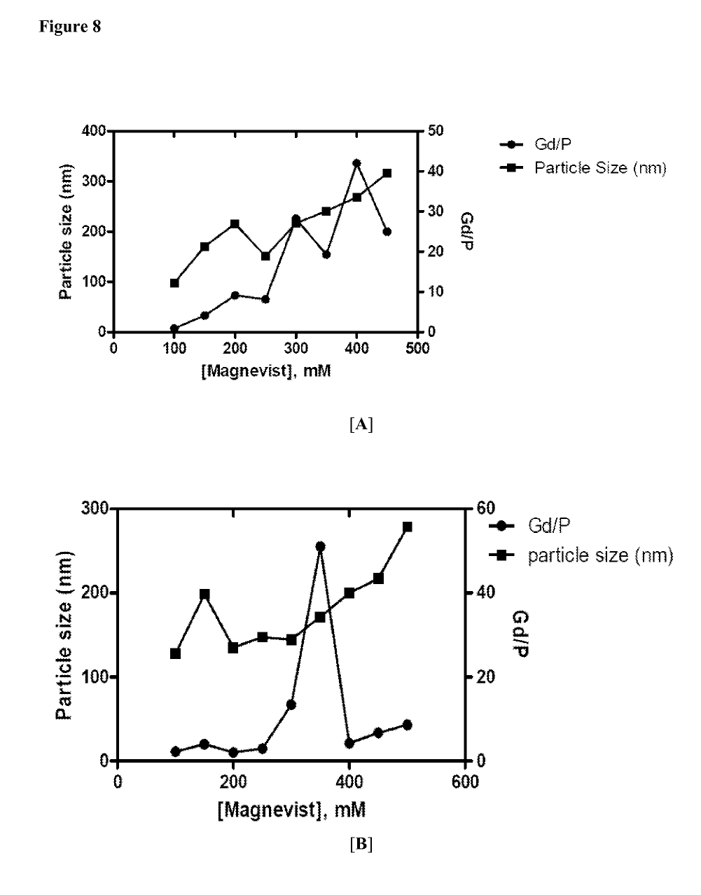

Low temperature sensitive liposomal compositions comprising Magnivest® and Doxorubicin (“Magnivest-Dox-LTSL”) were prepared as follows. Magnivest (469.01 mg/mL) was lyophilized and 157.3 mg was re-dissolved in 3 mL of citrate buffer (final pH=4.0, 52.5 mg/mL Magnivest). DPPC:MSPC:DSPE-PEG in molar % ratio of 85.3:9.7:5.0 were mixed and dissolved in chloroform and dried by solvent evaporation. The dried film (150 mg) was hydrated with different concentrations of Magnivest solution (100-500 mM) in citrate buffer (150 mM, 120 mM or 90 mM) at 60° C. for 15 min for citrate diluted contrast agent (FIG. 8a) or 55° C. for water diluted contrast agent (FIG. 8b), resulting in a lipid concentration or 50 mg/mL). The outside pH was neutralized with 0.5 M of Na₂CO₃ (2.2 mL) to obtain a pH of 7.47. Then, the resulting Multilamellar preparation was sized by repeated extrusion through Nuclepore® polycarbonate membrane filters (Whatman plc, UK) with a pore size of 100 nm using LIPEX™ Extruder (Northern Lipids Inc., Burnaby, Canada) at 55° C. Particle size of the liposome was determined by dynamic light scattering (ZetaPALS, Brookhaven Instruments Corporation, Holtsville, N.Y.).

For active Dox loading, 1.5 mL of Dox (5 mg/mL in 0.9% Saline, 0.5 mg of Dox per 100 mg of lipids) was added to the sized liposomes and the resulting mixture was incubated at 37° C. for 1 hr.

Sequential Active Loading of Dox and Gd into LTSL (Method II)

Low temperature sensitive liposomal compositions comprising gadolinium, DTPA and Doxorubicin (“Gd-DTPA-LTSL-Dox”) were prepared as follows. DPPC:MSPC:DSPE-PEG in molar % ratio of 86.3:9.7:5 were mixed and dissolved in chloroform and dried by solvent evaporation. The dried film (100 mg lipid) was hydrated with 2 mL hydrating buffer 1 (or 3 mL of hydrating buffer 2 or 3) to a final concentration of 50 mg lipid/mL then heated at 60° C. for 15 min. The resulting Multilamellar preparation was sized by repeated extrusion through Nuclepore® polycarbonate membrane filters (Whatman plc, UK) with a pore size of 100 nm using LIPEX™ Extruder (Northern Lipids Inc., Burnaby, Canada) at 55° C. to obtain unilamellar liposomes.

The outside pH of the sized liposomes was neutralized by buffer exchange with external buffer (250 mM sucrose, 20 mM HEPES, pH 7.4, 301 mOsm) by passing the lipid mixture through a Sephadex G-50 column equilibrated with the external buffer.

Then, 0.5 mL of Dox (10 mg/mL in deionized water, 5 mg of Dox per 100 mg of lipids) or 0.75 mL Dox (10 mg/mL in deionized water, 7.5 mg of Dox per 150 mg of lipids) was added in to the eluted lipid mixture and the resulting mixture was incubated at 37° C. for 1 hr.

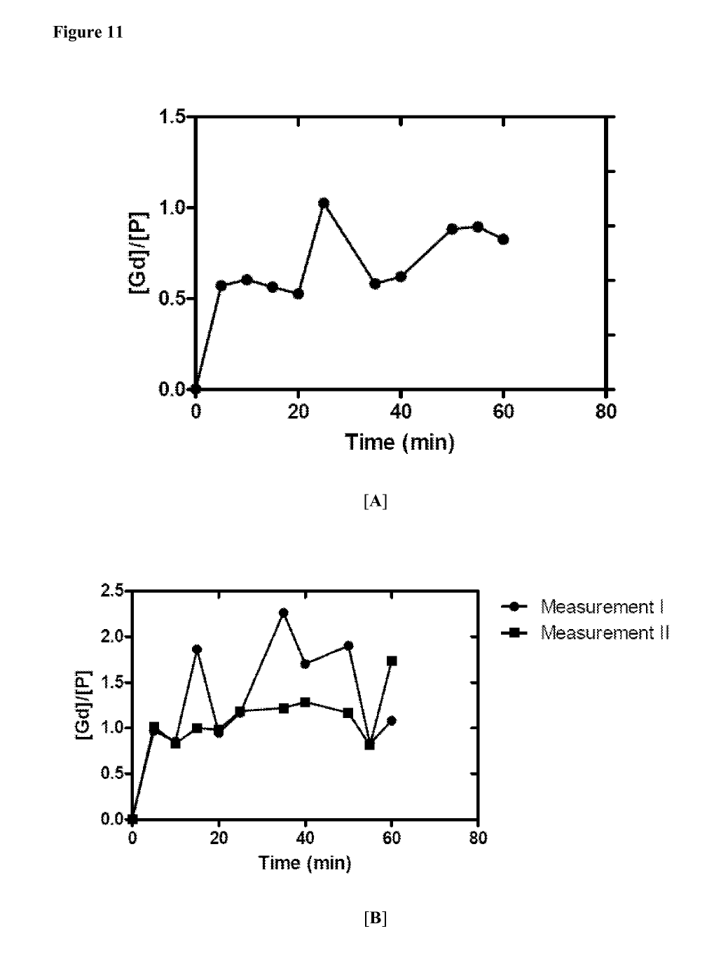

The Dox loaded LTSL was mixed with a film of A23187 ionophore (100 μg, 150 μg or 200 μg) and incubated at 37° C. for 20 min (FIG. 11).

Finally, 0.5 mL of GdCl₃ (aqueous) solution (180 mM) was added into the incubated mixture and incubation continued for additional 1 hr at 37° C.

### Measuring Gadolinium in Gd-DTPA-Dox-LTSL

300 μL of sample was withdrawn at 0, 10, 20, 30, 40, 50 and 60 min and spun in spin column (5 mL) packed with Sephadex G-50 equilibrated with 0.9% saline to trap the unencapsulated GdCl₃. The gadolinium content was measured using ICP-AES technique.

### Imaging Gd-DTPA-Dox-LTSL

LTSLs were used in preliminary imaging studies. Gd-DTPA-Dox containing LTSL (intact), Gd-DTPA-Dox containing LTSL (lysed) and half concentration of the lysed LTSL and de-ionized water (control metal free) were placed in NMR tubes and imaged at 7.0 T (Bruker 300 MHz NMR instrument), spin echo sequence: TR=50, 100, 200, 300, 500, 700, 1200, 3000, 5000, 7000 ms, TE=6 ms, number of signal averages=1,1 slice coronal (2 mm thick). FOV; 20×20 mm, matrix of 128×128.

### Determination of Gadolinium Concentration in Magnivest-Dox-LTSL

A calibration curve for gadolinium in Magnivest-Dox-LTSL was prepared as outlined below. 1 mL of Magnevist (469.01 mg/ml) diluted to 2 mL total volume was serially diluted 11 times to give a concentration of 234.5, 117.3, 58.6, 29.3, 14.7, 7.3, 3.6, 1.8, 0.92, 0.46 and 0.26 mg/mL. Si was calculated based on Si being equal to So(1-e(-TR/T₁). T₁ was measured for the concentration range of 3.6-0.26 mg/mL. 1/T₁ vs [Conc.] was plotted and the slope of the resulting line was determined. The concentration of gadolinium from the lysed LTSLs was then determined from the calibration curve.

### Results for Magnevist-Dox-LTSL and Gd-DTPA-Dox-LTSL

It was found that the Magnevist-Dox-LTSL particle size was 93.1±2.9. It was also found that the lysed Magnevist-Dox-LTSL had a greater signal than unlysed Magnevist-Dox-LTSL at the same concentration. Furthermore, it was found that the T₁ value decreased by about 50% when Magnevist-Dox-LTSL was lysed, as shown in Table 1.

TABLE 1
Table of T1 Values (Magnevist-Dox-LTSL)

| | T₁ (s) | 1/T₁ (s⁻¹) |
|---|---|---|
| LTSL (unlysed) | 0.107 | 9.412 |
| LTSL (lysed) | 0.055 | 18.07 |
| De-ionized water | 2.898 | 0.345 |

The final concentration of Magnevist was 4.66 mg/mL in the Magnevist-Dox-LTSL solution.

For Gd-DTPA-Dox-LTSL, it was found that the amount of Gd inside the LTSL increased with time as shown in Table 2.

TABLE 2
Loading Efficiency for Gd-DTPA-Dox-LTSL

| Time | [Gd] | | |
|---|---|---|---|
| point (min) | (mg/mL) | [P] (mg/mL) | [Gd]/[P] |
| 0 | 0.00238104 | 2.523961 | 0.0009434474 |
| 10 | 2.093452955 | 1.95593 | 1.070316063 |
| 20 | 2.51521514 | 1.998802 | 1.258361107 |
| 30 | 1.308093271 | 1.777962 | 0.735726047 |
| 40 | 3.197979561 | 2.401043 | 1.331912567 |
| 50 | 2.699710434 | 1.916117 | 1.408948647 |
| 60 | 3.781065724 | 1.957309 | 1.931767614 |

The theoretical maximum concentration of Gd inside the Magnevist-Dox-LTSL is about 90 mM and inside the Gd-DTPA-Dox-LTSL is about 45 mM, a 2:1 ratio. It was found that there was approximately twice the amount of Gd inside the Magnevist-Dox-LTSL (3.89 [Gd]/[P]) than the Gd-DTPA-Dox-LTSL (1.93 [Gd]/[P]) after 60 minutes, as should be expected.

### Preparation of Gd-HP-DO3A-Dox-LTSL (Method I)

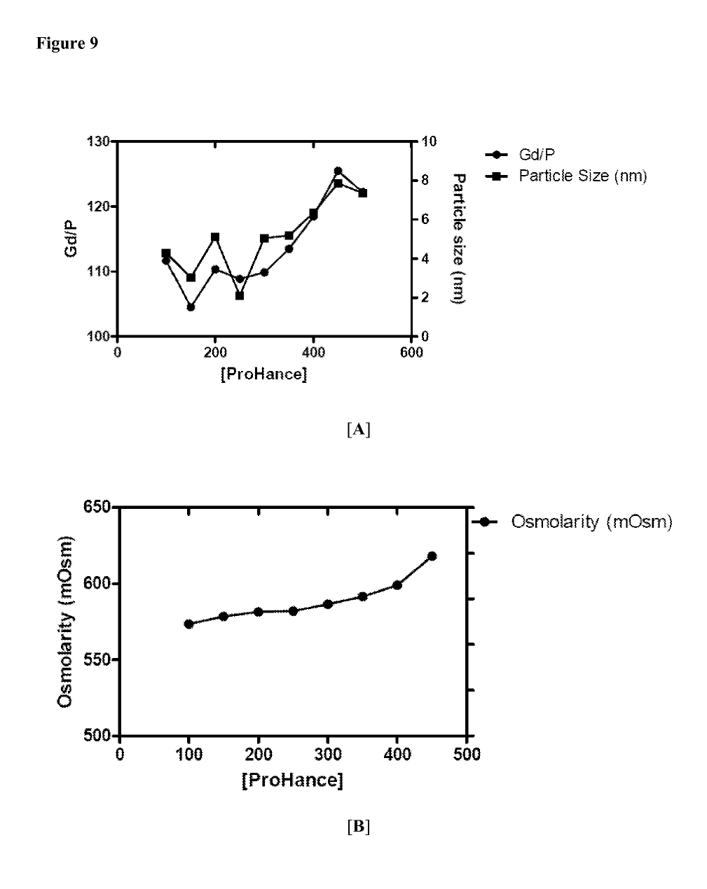

Liposomes were prepared by hydration of lipid film, followed by extrusion as previously reported (Mayer et al. (1985) Biochim Biophys Acta. 817(1):193-6). Briefly, lipid components (DPPC, MSPC and DSPE-PEG₂₀₀₀) were dissolved in chloroform at a molar ratio of 85.3:9.7:5.0. The solvent was evaporated using a Rotovap system and left overnight under a vacuum dessicator. The resulting lipid film was hydrated in hydrating buffer (300 mM Gd-HP-DO3A and 100 mM Citrate, mOsm=501-550, pH 4.0) at 60° C. for 15 minutes to yield a final lipid concentration of 50 mg/mL. Liposomes of approximately 100 nm diameter were obtained by extruding the mixture 5 times with a LIPEX™ Extruder (Northern Lipids Inc., Canada) at 55° C. through two stacked Nuclepore® polycarbonate membrane filters (Whatman PLC, United Kingdom) with a pore size of 100 nm (FIG. 9A).

Encapsulation of Dox into the extruded liposomes was carried out using a pH-gradient loading protocol as described by Mayer et al. (Mayer et al. (1986) Biochim Biophys Acta. 857(1):123-6) with a slight modification: exterior pH of the extruded liposomes was adjusted to 7.4 with sodium carbonate solution (500 mM) creating a pH gradient (acidic inside LTSL). The liposomes were incubated with Dox (Dox:lipid weight ratio of 5:100) at 37° C. for 1 h. Unencapsulated Gd-HP-DO3A and Dox were removed by passing the liposome through Sephadex-G50 (fine) spin column and the resulting liposomes were stored at 4° C.

Particle size of the liposome was determined by dynamic light scattering (Nanosizer, Malvern Instruments, USA) and reported as the mean diameter±standard deviation of 3 replicate dilutions. The concentration of liposome-entrapped Dox (Gd-HP-DO3A-Dox-LTSL) was determined using a UV-Vis spectrophotometer (PerkinElmer, USA) as previously reported (Fenske et al. Liposomes. 2nd ed: Oxford University Press; 2003). Concentrations of gadolinium and phosphorus were obtained by inductively coupled plasma-atomic emission spectroscopy (ICP-AES; Perkin-Elmer Plasma 40, USA), operated at the wavelengths of 342.247 and 213.617 nm for Gd and phosphorus detection, respectively (Molinelli et al. (2002) Inhal Toxicol. 14(10):1069-86). These measurements were reported as concentrations of Gd-HP-DO3A and as weight percent of Dox and gadolinium to lipid, where the lipid concentration was based on the phosphorus concentration.

Release of Dox and Gd-HP-DO3A from Gd-HP-DO3A-Dox-LTSL

Doxorubicin Release Quantification. The release of encapsulated Dox from Gd-HP-DO3A-Dox-LTSL as a function of temperature (25, and 37-41.3° C.) was determined by measuring de-quenching of Dox fluorescence as it was released from a liposome over a period of 15 minutes using Cary Eclipse spectrofluorimeter equipped with Eclipse multicell peltier, temperature controller, and Eclipse Kinetic Software (Varian Inc., City, Calif.) at an excitation and emission wavelengths of 498 and 593 nm, respectively. A sample of liposome was added into a cuvette containing 2 mL of HEPES buffer (pH 7.4, 10 mM, 280 mOsm) or human plasma equilibrated to the desired temperature. In order to compensate for lower fluorescence intensity of Dox in plasma, different amounts of Gd-LTSL-Dox were used in HEPES buffer (8 μL) and in plasma (70 4). Fluorescent intensity was measured every 7 s for 15 minutes. To obtain a measurement of fluorescence after complete release, surfactant was added (8 μL of 10% w/w and 70 μL of 25% w/w Triton® X-100 were used in HEPES and plasma experiments, respectively). Percent release is calculated by assuming 100% release with Triton® X-100 and 0% release at 25° C. in a HEPES buffer. Data are presented as the mean percent release (n=3). The same procedures were repeated to assess release at 25, 37, 40 and 41° C. after the liposome solution was stored at 4° C. for 7 days. The percent release of doxorubicin was calculated from equation (1):

% Release=100% (I₁−I₀/I_{f}−I₀) (1)

where Iₜ represents the fluorescence intensity at time t, I₀ is the fluorescence intensity at 25° C.; and I_{f} is the intensity after the addition of Triton X-100.

Gd-HP-DO3A Release Quantification. Release of Gd-HP-DO3A was quantified using two methods: 1) Measurement of longitudinal relaxation time at 0.5T and the use of a calibrated relationship between 1/T1 and contrast agent concentration and 2) ICP-AES measurements of Gd concentration. For the T1 method, samples obtained during the release assay were further diluted using HEPES buffer and their T1 of was measured using a custom-built relaxometer. They were then passed through Sephadex-G50 (fine) spin columns twice to remove released contrast agent. Triton X-100 was added to each sample and T1 was once again measured. In order to convert the T1 reading to Gd-HP-DO3A concentration, a calibration curve was developed relating 1/T1 to Gd-HP-DO3A concentration (0-4 mM). The same samples whose T1 was measured above were then analyzed for Gd and phosphorus content using ICP-AES.

Percent release of Gd-HP-DO3A was calculated using both T1-based measurements of Gd-HP-DO3A and Gd concentration obtained with ICP-AES. Equation (2) used to calculate percent release was modified to account for the fact that released Gd-HP-DO3A would be removed by the spin columns:

% Release=100% (C₀−Cₜ/C₀−C_{T}) (2)

where Cₜ is the concentration of contrast agent released from the liposome at time t; C₀ is the concentration at time equals zero, before release occurs; and C_{T} is the concentration of the sample treated with Triton X-100, before being passed through the columns.

Analysis of liposome content release kinetics. A least squares fit of Equation (3):

% Release=Max (1−e^{-Kt}) (3)

to release curves for Dox and Gd-HP-DO3A was used to produce estimates of release rates (K), maximum release (Max), and the time to 50% release (ln(2)/K).

### Thermoscan Procedure

Doxorubicin release from Gd-HP-DO3A-Dox-LTSL was also studied by measuring fluorescence as a function of temperature. A quartz cuvette with 10 μL Gd-HP-DO3A-Dox-LTSL in 2 mL HEPES solution was heated at a ramp-up rate of 1° C./min from 20° C. to 55° C. and subsequently cooled at the same rate to 20° C. Fluorescence readings were taken every 30 seconds. Triton® X-100 (10 μL, 10% w/w in deionized water) was added to a second cuvette with the same concentration of LTSL as a control. A third cuvette holder in the Peltier unit was used for temperature feedback by the instrument. The three cuvette holders were within 0.1° C. in this temperature range. The highest permeability of the LTSL membrane to Dox was defined as the maximum of the derivative of the resulting curve.

### Gd-HP-DO3A-Dox-LTSL Stability

Liposome stability was investigated by measuring their size for 7 consecutive days, and release of Dox in HEPES buffer (at 25, 37, 40 and 41° C.) at days 0 and 7. Day zero is defined as immediately after the Gd-HP-DO3A-Dox-LTSL was made and the Gd-HP-DO3A-Dox-LTSL was stored at 4° C.

### Measurement of Relaxivity of Gd-HP-DO3A-Dox-LTSL

Gd-HP-DO3A was released from Gd-HP-DO3A-Dox-LTSL (0.016 to 2.0 mM Gd-HP-DO3A) by heating above the Tm using a hot water bath (55° C. for 10 min). T₁ of all of the solutions was calculated from the fit of signal intensity vs. inversion time in images obtained with a T₁-weighted inversion recovery sequence with variable inversion time (T₁=50, 75, 100, 150, 300, 450, 600, 900, 1050 ms). Relaxivity was obtained as the slope of 1/T1 vs. Gd-HP-DO3A concentration. All imaging was performed using a clinical 1.5T MR scanner (Philips Medical Systems, Best, The Netherlands).

### Phantom Preparation Using Gd-HP-DO3A-Dox-LTSL

Tissue mimicking agar-silica phantoms containing Gd-HP-DO3A-Dox-LTSL were prepared using silicone powder and agarose powder (2 wt % of each each). These were mixed in 290 mOsm HEPES buffer (pH=7.4) and heated above to above 90° C. for 30 minutes while constantly mixing. Two different phantoms were constructed. One phantom had cavities cast inside the gel while solidifying that were later loaded with Gd-HP-DO3A-Dox-LTSL solutions. The other phantom was cast with a large rectangular cavity that was then filled with a similar tissue mimicking composition but using low melting agarose and Gd-HP-DO3A-Dox-LTSL to make a continuous region of Gd-HP-DO3A-Dox-LTSL This low melting agarose solution was allowed to cool to 35° C., at which point Gd-HP-DO3A-Dox-LTSL was added, while mixing to prevent liposome release, to result in an approximate concentration of 0.2 mM Gd-HP-DO3A.

### MR-HIFU Procedure Using Gd-HP-DO3A-Dox-LTSL

The Philips MR-HIFU treatment system integrates an ultrasound transducer with MR-imaging and electromechanical transducer positioning system, delivering spatially and temporally controlled ultrasound energy. The MR system is used to plan the therapy with 3D anatomical imaging and to guide and monitor hyperthermia with thermal imaging during treatment. Heating with MR-HIFU was achieved by focusing an ultrasound beam using a 256-element phased array focused piezoelectric ultrasound transducer immersed in a sealed tank of degassed water, running at 1.2 MHz. A single 2 mm HIFU focus was steered electronically (by altering the phases of the elements) in concentric circles (Salomir et al. (2000) J Magn Reson Imaging 12(4):571-83).

Feedback control of MR-HIFU MR thermometry to control the HIFU exposure during heating using temperature calculated using the PRFS method (Hindman et a;. (1966) J. Chem. Phy. 44(12):4582-9). Temperature was raised with constant acoustic power until the mean temperature of the treatment cell increased above the cutoff temperature (T>42.0° C.). The treatment cell was then allowed to cool for a fixed period of time (30 sec). This heat and cool cycle was repeated to achieve the desired duration of hyperthermia.

Two scans were run on the MR scanner: a planning sequence before the treatment and a temperature monitoring sequence during the treatment. T₂-weighted turbo spin-echo (TSE) images were acquired as a 3D coronal stack, transferred to the workstation, and used for ultrasound exposure planning To monitor the induced temperature elevation during each sonication, a multi-shot T₁-weighted FFE-EPI sequence was performed every 2.9 seconds. The agar-silica-gel phantom with suspended Gd-HP-DO3A-Dox-LTSL was positioned on the treatment table, and acoustic coupling was achieved using degassed water.

### Statistical Analysis

Fitted parameters were compared using the F-test. Differences in percent release were compared using Dunn's multiple comparison test. Error reported for interpolated values was estimated as SEM of replicate experiments. All fitting and statistical analysis was performed using GraphPad Prism (version 5.00 for Windows, GraphPad Software, San Diego Calif. USA, www.graphpad.com). Results were considered significant with p<0.05, and two-tailed p-values were obtained in all cases. Pairwise comparisons with Dunn's multiple comparison test were only reported when Kruskal-Wallis showed significant differences between all tested groups.

Release of Dox from Gd-HP-DO3A-Dox-LTSL

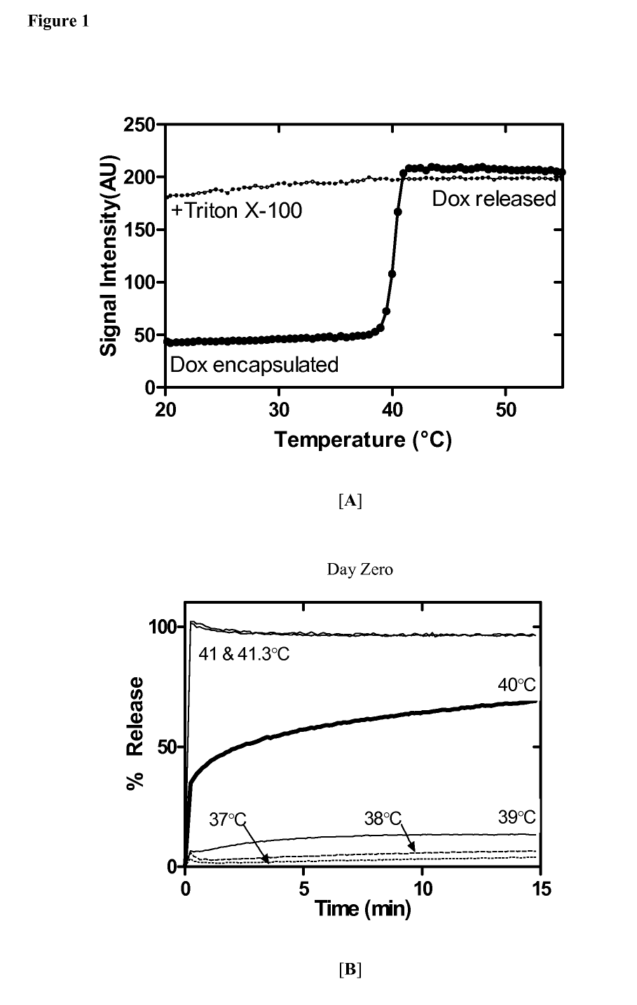

Dox release. Using fluorescence dequenching, the release of doxorubicin from Gd-HP-DO3A-Dox-LTSL was measured in HEPES buffer. FIG. 1A shows that doxorubicin fluorescence increases gradually as temperature increases before rapid drug release occurs around the temperature of peak permeability of the liposomal membrane (solid-to-gel transition temperature). The plateau above the melting temperature describes a combination of processes, where the release is slower due to lower liposome membrane permeability as well as the lower concentration of doxorubicin in the liposome since much of the doxorubicin release already occurred. As shown in FIG. 1B, doxorubicin release from the Gd-HP-DO3A-Dox-LTSL is minimal at 37-39° C. for 15 minutes. At 40° C. there was about 30% instantaneously released followed by a more gradual release. Near the Tm, complete release occurs in less than 24 sec at 41° C. and 41.3° C.

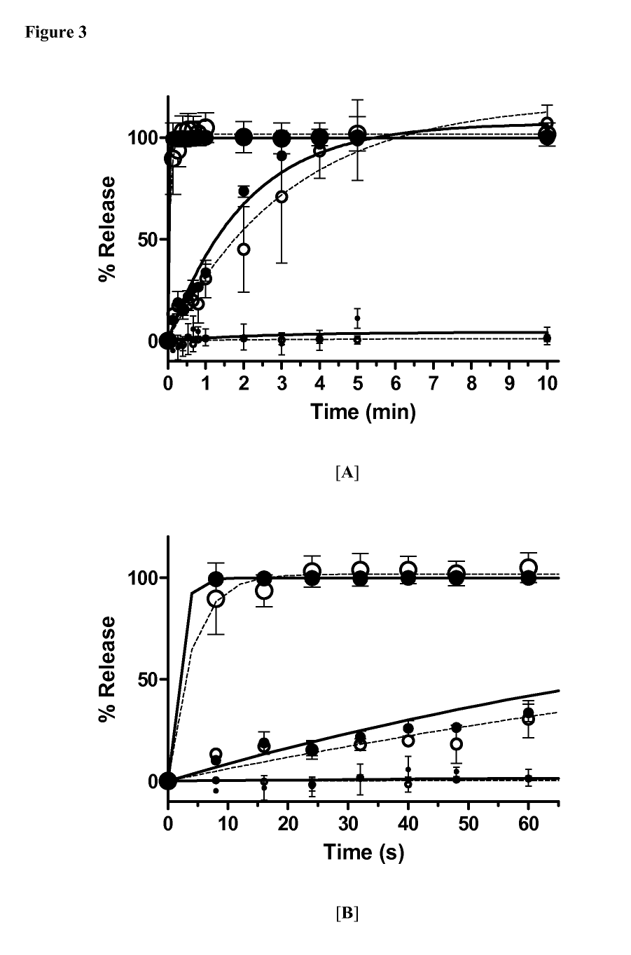

Gd-HP-DO3A release. Release of Gd-HP-DO3A was measured and compared to Dox release using Dox dequenching in the same sample as shown in FIG. 3A. The difference in percent release between amounts of Dox and Gd-HP-DO3A release is less than 20% for all time points, with the exception of 2 time points at 40° C. Mean absolute differences between amounts of Dox and Gd-HP-DO3A released are not significant (p>0.05, Dunn's multiple comparison). This lack of difference in release is especially evident in the first minute of release (FIG. 3B). The rates of Dox and Gd-HP-DO3A release are shown in Table 3 along with other results of the fitting procedure for comparison. The release relaxivity of heated and unheated liposomes was measured as shown in FIG. 2. The relaxivity significantly increased when a Gd-HP-DO3A-Dox-LTSL solution was heated.

TABLE 3
Rates of Dox and Gd-HP-DO3A release from Gd-HP-DO3A-Dox-LTSL.
Fitted values are reported with their standard errors or confidence
intervals, where appropriate.

| | 37° C. | 40° C. | 41.3° C. |
|---|---|---|---|
| Doxorubicin | | | |
| Release Rate Constant (s−1) | 0.4 ± 1.2 | 0.52 ± 0.08 | 250 ± 40 |
| Maximum Release (%) | 1.2 ± 1.5 | 117 ± 9 | 101.6 ± 1.3 |
| R² | 0.049 | 0.889 | 0.936 |
| ProHance ® | | | |
| Release Rate Constant (s−1) | 0.6 ± 1.6 | 0.82 ± 0.06 | 64 ± 3 |
| Maximum Release (%) | 4 ± 5 | 107 ± 3 | 99.82 ± 0.04 |
| R² | 0.049 | 0.967 | 0.999 |
| Absolute Average Difference | 2.8 ± 1.5 | 6 ± 4 | 3 ± 2 |
| (%) | | | |

The rate of release of Dox from Gd-HP-DO3A-Dox-LTSL in plasma was also studied in a similar manner to that in HEPES buffer. The extent of drug release after 10 min at 37-39° C. in plasma was high compared to release of Dox in HEPES to the corresponding temperature points. However, the release of Dox was substantially lower at and above 40° C. in plasma as compared to in HEPES for similar temperature points, where we observed near to complete release. These observations were also confirmed from the thermoScan experiments (results not shown).

### Stability of Gd-HP-DO3A-Dox-LTSL

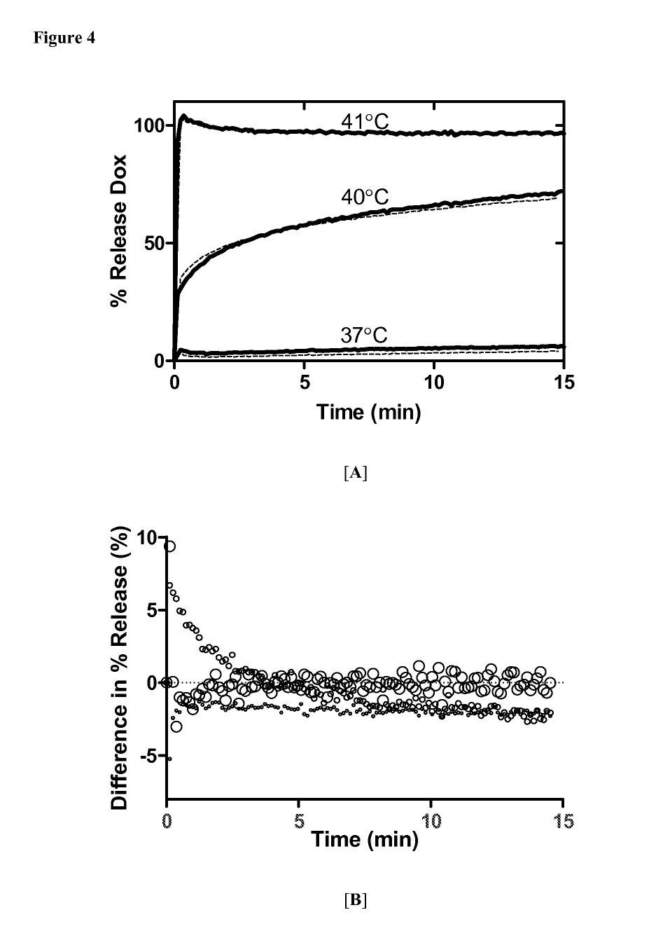

Liposome stability, indicated by size (97.4±0.6 nm, n=7), was relatively constant for one week. The rates of Dox release also remained similar a week after synthesis and storage at 4° C. of Gd-HP-DO3A-Dox-LTSL (FIG. 4A). Overall there was slightly less release on day 7 (median decrease in release of 0.13-1.9%), as indicated by the difference in percent release (FIG. 4B). This difference was significant at 37° C. (p<0.05, Dunn's multiple comparison test), but not at other temperatures (p>0.05).

Triggered Release from Gd-HP-DO3A-Dox-LTSL with MR-HIFU

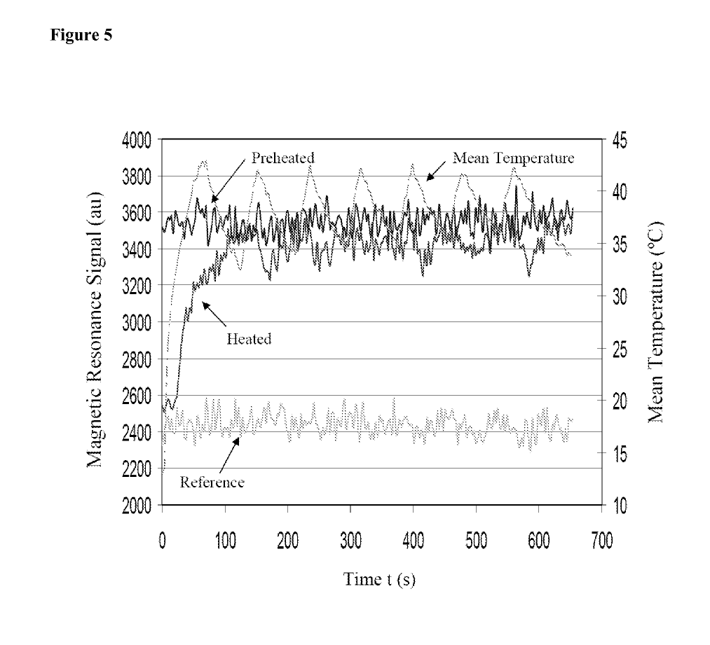

An agar-silica-gel phantom with suspended LTSL was heated with mild hyperthermia using MR-HIFU. The heating was fairly homogeneous with a flat profile above 40° C. extending 13 mm in the coronal plane. The temperature fluctuated with the HIFU exposures as shown in FIG. 5. The temperature increased during the sonication and decreased when no power was applied. The duration of each cooling cycle was 30 sec, which precisely corresponds to the duration of the cooling cycle in FIG. 5. During and after the sonication, a signal enhancement of 40% was caused by Gd-HP-DO3A release from Gd-HP-DO3A-Dox-LTSLs. The signal intensity of a volume that was pre-heated remained the same throughout the sonication, and it was consistently higher than the signal intensity of the region that was not heated.

### REFERENCES

The references listed below as well as all references cited in the specification are incorporated herein by reference to the extent that they supplement, explain, provide a background for or teach methodology, techniques and/or compositions employed herein.

Abraham et al. (2002) Biochim. Biophys. Acta 1565: 59-72

Allen & Cullis (2004) Science 303:1818-22.

Allen, T. M. (1998) Drugs 56(5):747-56.

Allen, T. M. (2002) Nat Rev Cancer 2(10):750-63.

al-Shabanah et al. (1994) Chemotherapy 40:188-94.

Anyarambhatla & Needham (1999) J Liposome Res 9:491-506.

Arap et al. (1998) Science 279(5349):377-80.

Baba (1993) Anticancer Res 13:651-4.

Bacic et al. (1988) Magn. Reson. Med. 6(4): 445-58.

Bartlett (1959) J Biol Chem 234:466-8.

Bogdanov et al. (1993) Radiology 187:701.

Bondurant et al. (2001) Biochimica et Biophysica Acta 1511:113-122.

Caravan et al. (1999) Chemical Reviews 99(9):2293-352.

Chelvi et al. (1995) Oncol Res. 7(7-8):393-8.

Chen et al. (2004) MoI Cancer Ther 3 1311-7.

Cheung et al. (1998) Biochim Biophys Acta 1414(1-2):205-16.

Cope et al. (1990) Cancer Res 50: 1803-9.

Cummings (1985) J Chromatog 341:401-9.

Dessler et al (1994) Invest. Radiol. 29 supl. 2:S65.

Dewhirst et al. (2003) Hyperthermia. In: Bast R et al., editors. Cancer Medicine. 6th ed. Hamilton, Ontario:B.C. Decker Inc.

Dromi et al. (2007) Clinical Cancer Research 13(9):2722-7.

Drummond (1999) Pharmacol Rev 51:691-743.

Fan et al., (1999) J. MoI. Med. 77:577-596.

Fenske et al. (2003) Liposomes. 2nd ed: Oxford University Press.

Fiske & Subbarow (1925) J Biol Chem 2:375-95.

Fornasiero et al. (1987) Invest Radiol 22:322-7.

Fossheim et al. (1999) Magn Reson Imaging 17:83-9.

Fossheim et al. (2000) Acad Radiol 7: 1107-15.

Frei & Eder (2003) Principles of dose, schedule, and combination therapy. In: Bast et al., editors. Cancer Medicine. 6th ed. Hamilton, Ontario: B.C. Decker Inc.

Gabizon et al. (1990) Cancer Res 50:6371-8.

Gabizon et al. (1991) Br J Cancer 64: 1125-32.

Ghaghada et al. (2007) American Journal of Neuroradiology 28(1):48-53.

Ghaghada et al. (2008) Academic Radiology 15(10):1259-63.

Ghaghada et al. (2009) PLoS ONE 4(10):e7628. doi:10.1371/journal.pone.0007628

Glogard et al. (2002) Int J Pharm 233:131-40.

Glogard et al. (2003) Magnetic Resonance in Chemistry 41(8):585-8.

Goins & Phillips (2001) Prog Lipid Res 40:95-123.

Grant & Wells (1974) J Surg Res 16:533-40.

Gregoriadis et al. (1980) Receptor-mediated Targeting of Drugs. Plenum Press, New York, pp. 243-266.

Hahn et al. (1975) Proc Natl Acad Sci USA 72:937-40.

Hak et al. European Journal of Pharmaceutics and Biopharmaceutics 72(2):397-404.

Harrison's Principles of Internal Medicine (Fauci et al., Eds.) 14th Ed. McGraw-Hill, New York, p. 84 (1998)

Hauck et al. (2006) Clin Cancer Res. 12(13):4004-10.

Hindman et al. (1966) J. Chem. Phys. 44(12):4582-92.

Hynynen et al. (1993) Med Phys. 20(1):107-15.

Kakinuma et al. (1996) Int. J. Hyperthermia 12(1): 157-165.

Kanal et al. (2007) American Journal of Roentgenology 188(6):1447-74.

Karathanasis et al. (2008) Nanotechnology 19(31):5101-9.

Kawai et al. (1997) Cancer 79:214-9.

Kean & Smith (1986) Magnetic Resonance Imaging: Principles and Applications. Williams and Wilkins, Baltimore, Md.

Knight (1981) Liposomes: From physical structure to therapeutic applications. Elsevier, North Holland, pp. 310-311.

Kong & Dewhirst (1999) Int. J. Hyperther. 15(5): 345-70

Kong et al. (2000) Cancer Res 60:4440-5.

Kong et al. (2000) Cancer Res 60:6950-7.

Kong et al. (2001) Cancer Res 61:3027-32.

Koning & Krijger (2007) Anti-Cancer Agents in Medicinal Chemistry 7:425-40.

Koukourakis et al. (1999) J Clin Oncol 17:3512-21.

Krauze et al. (2005) Brain Res Brain Res Protoc 16:20-6.

Krauze et al. (2005) Exp Neurol 196:104-11.

Kusaka et al. (1992) Magn Reson Med 24:137-48.

Laurent et al. (2008) Langmuir 24(8):4347-51.

Litzinger & Huang, (1992) Biochim Biophys Acta 1113: 201-227.

Lokling et al. (2001) Magn Reson. Imag. 19:731-8.

Lokling et al. (2003) Magn. Reson. Imag. 21:531-540.

Lokling et al. (2004) J Control Release 98:87-95.

Lokling et al. (2004) Mag Reson in Med 51(4):688-96.

Lyng et al. (1998) Magn. Reson. Med. 40(1):89-98.

Maeda H. (2001) Adv Enzyme Regul. 41:189-207.

Magin & Niesman (1984) Cane. Drug Del. 1(2):109-17.

Mayer et al. (1985) Biochim Biophys Acta 17(1):193-6.

Mayer et al. (1985) J. Biol. Chem. 260(2):802-808.

Mayer et al. (1986) Biochim Biophys Acta 857(1):123-6.

McDannold et al. (2004) Radiology 230:743-52.

Meyer et al. (1994) Invest. Radiol. 29 supl. 2:S90.

Mills & Needham (2005) Biochim Biophys Acta 1716:77-96.

Modi et al. (2006) Current Pharmaceutical Design 12(36):4785-96.

Molinelli et al. (2002) Inhal Toxicol. 14(10):1069-86.

Mueller et al. (2000) Macromolecules 33:4799-4804.

Mulder et al. (2004) Bioconjugate Chemistry 15(4):799-806.

Naoi & Yagi (1984) Biochem. Int. 9:267-272.

Needham et al. (2000) Cancer Res. 60(5):1197-201.

Newman et al. (1992) Int J Hyperthermia 8:79-85.

Ni et al. (1997) Acta Radiol 38(4 Pt 2):700-7.

Niesman et al. (1990) Invest. Radiol. 25(5): 545-51.

Ogan et al. (1987) Invest. Radiol. 22:665.

Pak et al. (1999) Biochimica Et Biophysica Acta-Biomembranes 1419(2):111-26.

Papahadjopolous (1979) Ann. Rep. Med. Chem. 14:250-260

Park et al. (2002) Clin Cancer Res 8:1172-81.

PCT Publication Nos. WO92/21017; and WO08/82657; both of which are hereby incorporated by reference in their entireties.

Phillips & Goins (1995) Targeted delivery of imaging agents by liposomes. Torchilin V, editor. Boca Raton, Fla.: CRC Press.

Ponce et al. (2005) Monitoring of liposomal drug distribution in vivo using MRI. In: Era of Hope Conference for Congressionally Funded Breast Cancer Research 2005 Philadelphia.

Ponce et al. (2006) Future Lipidology 1 :25-34.

Ponce et al. (2007) J Natl Cancer Inst. 99(1):53-63.

Poon et al. (2009) Expert Opin Pharmacother. 10(2):333-43.

Port et al. (2006) Cancer Chemother Pharmacol 58:607-17.

Port et al. (2008) Biometals 21(4):469-90.

Qiu et al. (1997) IEEE Trans. Biomed. Eng. 44(11):1107-1113

Rubin & Hait (2003) Anthracyclines and DNA intercalators. Cancer Medicine, 6th Ed. BC Decker, Inc.

Saito et al. (2004) Cancer Res 64:2572-9.

Saito et al. (2005) Exp Neurol 196:381-9.

Salomir et al. (2005) J Magn Reson Imaging 22:534-40.

Salomiret al. (2000) J Magn Reson Imaging. 12(4):571-83.

Sarkar et al. (2005) Chemical Communications 999-1001.

Schroeder et al. (2009) Journal of Controlled Release 137(1):63-8.

Schroedera et al. (2009) Chemistry and Physics of Lipids 162(1-2):16.

Schumann-Giampieri et al. (1991) Invest. Radiol. 26:969.

Schwendener et al. (1990) Invest. Radiol. 25(8): 922-32.

Seltzer, S. E. (1989) Radiology 171(1):19-21.

Senior et al. (1985) Biochim. Biophys. Acta 839:1-8.

Shen et al. (1994) Invest. Radiol. 29 supl. 2: S217.

Simoes et al. (2004) Advanced Drug Delivery Reviews 56(7):947-65.

Spratt et al. (2003) Biochimica et Biophysica Acta 1611:35-43.

Suga et al. (2001) Invest. Radiol. 36(3):136-45.

Surolia & Bachhawat (1977) Biochim. Biophys. Acta 497:760-765.

Takehara et al. (2001) Br. J. Cancer 84(12):1681-5.

Tilcock (1999) Adv Drug Deliv Rev 37(1-3):33-51.

Tilcock et al. (1989) Radiology 171(1):77-80.

Torchilin, editor (1997) Surface-modified liposomes in gamma- and MR-imaging.

Torchilin (2005) Nat Rev Drug Discov. 4(2):145-60.

U.S. Patent Application Publication No. 2009/0117035; hereby incorporated by reference in its entireties.

U.S. Pat. Nos. 5,094,854; 5,464,696; 5,525,232; 5,833,948; 5,888,476; 5,925,987; 5,961,953; 6,010,681; 6,200,498; and 6,200,598; all of which are hereby incorporated by reference in their entireties.

Unger et al. (1993) J. Mag. Res. Imag. 3:195-198.

Vexler et al. (1994) Invest. Radiol. 29 supl. 2:S62.

Viglianti et al. (2004) Magn Reson Med 51:1153-62.

Viglianti et al. (2006) Magn Reson Med 56:1011-8.

Wang et al. (1990) Radiology 175:483.

Wehrli et al. (1988) Biomedical magnetic resonance imaging: principles. methodology, and applications. John Wiley and Sons Ltd. New York.

Weinstein et al. (1979) Science 204(4389):188-91.

Winterhalter et al., editors (1997) Stealth® liposomes: From theory to product.

Yatvin et al. (1978) Science 202:1290.

Yuan et al. (1994) Cancer Res. 54:3352-56.

Yuan et al. (1996) Cancer Res. 55:3752-56.

Zhang et al. (2004) Pharmacological Research 49(2):185-98.

Zucker et al. (2009) Journal of Controlled Release 139(1):73-80.

It will be understood that various details disclosed herein may be changed without departing from the scope of the disclosure. Furthermore, the foregoing description is for the purpose of illustration only, and not for the purpose of limitation.

1. A method comprising the steps of:
reconstituting liposome-forming lipids with a solution comprising a contrast agent and a hydrating buffer, wherein the hydrating buffer has an osmolarity of between about 300 mOsm and about 700 mOsm;
incubating the pre-liposome solution at a temperature for a time; and
extruding the incubated solution through a filter, thereby forming a liposome.

2. The method of claim 1, wherein the liposome-forming lipids comprise phospholipids.

3. The method of claim 1, wherein the liposome-forming lipids comprise phosphatidylcholines.

4. The method of claim 1, wherein the liposome-forming lipids comprise phosphatidylcholines selected from the group consisting of dipalmitoylphosphatidylcholine (DPPC), distearoylphosphatidylcholine (DSPC), monostearoylphosphatidylcholine (MSPC), diarachidoylphosphatidylcholine (DAPC), and mixtures thereof

5. The method of claim 1, wherein the liposome-forming lipids comprise dipalmitoylphosphatidylcholine, monostearoylphosphatidylcholine, and 1,2-distearoyl-sn-glycero-3-phosphoethanolamine-N-[Amino(Polyethylene Glycol)2000].

6-9. (canceled)

10. The method of claim 1, wherein the contrast agent comprises an element selected from the group consisting of Gd, Cu, Cr, Fe, Co, Er, Ni, Eu, Dy, Zn, Mg, Mo, Li, Ta, and Mn.

11-16. (canceled)

17. The method of claim 1, wherein the hydrating buffer comprises citrate.

18-21. (canceled)

22. The method of claim 1, further comprising the steps of:
neutralizing the outside pH of the liposome; and
contacting the neutralized liposome with a compound under conditions wherein the compound is encapulsated by the liposome.

23. The method of claim 22, wherein the compound is a chemotherapeutic agent.

24-28. (canceled)

29. A method comprising the steps of:
reconstituting liposome-forming lipids with a solution comprising a chelating agent and a hydrating buffer;
incubating the pre-liposome solution at a temperature for a time;
extruding the incubated solution through a filter, thereby forming a liposome comprising the chelating agent;
contacting the liposome comprising the chelating agent with an external buffer, thereby neutralizing the outside pH of the liposome;
contacting the neutralized liposome with a compound under conditions wherein the compound is encapulsated by the liposome, thereby forming a liposome comprising the compound and the chelating agent; and
contacting the liposome comprising the compound and the chelating agent with an ionophore and a metal ion, under conditions where the ionophore assists in the encapuslation of the metal ion by the liposome comprising a compound and a chelating agent, thereby forming a liposome.

30. The method of claim 29, wherein the liposome-forming lipids comprise phospholipids.

31. The method of claim 29, wherein the liposome-forming lipids comprise phosphatidylcholines.

32. The method of claim 29, wherein the liposome-forming lipids comprise phosphatidylcholines selected from the group consisting of dipalmitoylphosphatidylcholine (DPPC), distearoylphosphatidylcholine (DSPC), monostearoylphosphatidylcholine (MSPC), diarachidoylphosphatidylcholine (DAPC), and mixtures thereof.

33. The method of claim 29, wherein the liposome-forming lipids comprise dipalmitoylphosphatidylcholine, monostearoylphosphatidylcholine, and 1,2-distearoyl-sn-glycero-3-phosphoethanolamine-N-[Amino(Polyethylene Glycol)2000].

34-37. (canceled)

38. The method of claim 29, wherein the chelating agent is diethylene triamine pentaacetic acid (DTPA).

39-41. (canceled)

42. The method of claim 29, wherein the hydrating buffer comprises citrate.

43-50. (canceled)

51. The method of claim 29, wherein the compound is a chemotherapeutic agent.

52. (canceled)

53. The method of claim 29, wherein the ionophore is an ionophoretic antibiotic.

54. (canceled)

55. The method of claim 29, wherein the metal ion comprises an element selected from the group consisting of Gd, Cu, Cr, Fe, Co, Er, Ni, Eu, Dy, Zn, Mg, Mo, Li, Ta, and Mn.

56. (canceled)

57. A liposome prepared by the method of any one of claims 1-5, 10, 17, 22, 23, 29-33, 38, 42, 51, 53 and 55.

58. A method of predicting efficacy of a treatment in a subject, the method comprising: administering to the subject a composition comprising a liposome of claim 57, provided the liposome comprises a compound; monitoring accumulation of the compound at a desired site in vivo by magnetic resonance imaging; and predicting efficacy of treatment based on accumulation of the compound at the desired site.

59. (canceled)

60. A method of targeting delivery of a compound of interest at a desired site in vivo, the method comprising: administering to a subject a composition comprising a liposome of claim 57, provided that the liposome comprises a compound, wherein a non-physiological environmental condition is present at the desired site, and the composition is targeted to a desired location at the desired site in the subject, at a desired rate of accumulation at the desired site, or both a desired location and a desired rate of accumulation at the desired site by the presence of the non-physiological environmental condition.
